(19) United States
(12) Patent Application Publication
(10) Pub. No.: US 2024/0366784 A1
(43) Pub. Date: Nov. 7, 2024

(54) INJECTABLE HYDROGEL COMPOSITIONS AND METHODS OF USE THEREOF

(71) Applicant: Drexel University, Philadelphia, PA (US)

(72) Inventors: Christopher B. Rodell, Cherry Hill, NJ (US); Shreya S. Soni, Burlington, NJ (US)

(21) Appl. No.: 18/654,097

(22) Filed: May 3, 2024

Related U.S. Application Data
(60) Provisional application No. 63/463,575, filed on May 3, 2023.

(51) Int. Cl. A61K 47/69; A61K 9/06; A61P 29/00

(52) U.S. Cl. CPC A61K 47/6939; A61K 9/06; A61K 47/6951; A61P 29/00

(57) ABSTRACT

The present disclosure relates, in one aspect, to hydrogel compositions comprising a nanoparticle, functionalized polymer, and a therapeutic agent. In certain embodiments, the hydrogel compositions of the disclosure are suitable for local delivery of a therapeutic agent.

### CROSS-REFERENCE TO RELATED APPLICATIONS

This application claims priority under 35 U.S.C. § 119(e) to U.S. Provisional Patent Application No. 63/463,575, filed May 3, 2023, which is incorporated herein by reference in its entirety.

### STATEMENT REGARDING FEDERALLY SPONSORED RESEARCH OR DEVELOPMENT

This invention was made with government support under 5R35GM147184-02 awarded by National Institutes of Health. The government has certain rights in the invention.

### BACKGROUND

Systemic drug administration is often hampered by biodistribution challenges, including rapid renal clearance, low or no tissue distribution specificity, and poor aqueous solubility of many small molecule drugs. Local delivery strategies mitigate these issues by minimizing drug exposure in off-target tissues and extending the window of therapeutically active drug concentrations at the site of action. Hydrogels therefore continue to evolve as tailorable platforms for therapeutic delivery that can support tissue regeneration, alter cell or tissue responses, or present appropriate therapeutic cargo. Such cargo may include pharmacologic drugs, exosomes, or cells intended to support processes of immune modulation and tissue repair. In particular, small molecule drugs are attractive payloads due to their ease of synthesis and ability for optimization; however, their local delivery from hydrogels is often challenging due to their rapid diffusive release and non-specific cellular biodistribution.

Thus, there is a need in the art for hydrogel compositions and methods of use thereof for local and sustained release of therapeutic cargo (e.g., small molecules). The disclosure addresses this need.

### BRIEF SUMMARY

In one aspect, the disclosure provides a hydrogel composition. In certain embodiments, the hydrogel composition comprises a nanoparticle comprising a plurality of macrocycles, wherein each of the macrocycles are covalently linked, either directly or indirectly, by at least one nanoparticle linker. In certain embodiments, the hydrogel composition comprises a functionalized polymer comprising a hydrophilic polymer core substituted with a plurality of hydrophobic substituents, wherein at least a portion of the hydrophobic substituents are independently non-covalently associated with one of the macrocycles of the nanoparticle. In certain embodiments, the hydrogel composition comprises at least one therapeutic agent, wherein the at least one therapeutic agent is non-covalently associated with a macrocycle of the nanoparticle.

In another aspect, the disclosure provides a method for treating, preventing, and/or ameliorating an inflammatory disease or disorder in a subject, the method comprising administering to the subject the hydrogel composition of the disclosure.

In another aspect, the disclosure provides a method for delivering a therapeutic agent to a macrophage in a subject, the method comprising administering to the subject the hydrogel composition of the disclosure.

In another aspect, the disclosure provides a method for reducing or inhibiting pro-inflammatory (M1-like) behavior of a macrophage in a subject, the method comprising administering to the subject the hydrogel composition of the disclosure.

In another aspect, the disclosure provides a method for promoting pro-healing (M2-like) behavior of a macrophage in a subject, the method comprising administering to the subject the hydrogel composition of the disclosure.

### BRIEF DESCRIPTION OF THE FIGURES

The drawings illustrate generally, by way of example, but not by way of limitation, various embodiments of the present application.

FIG. 1 provides a schematic depicting macrophage (MF) transition across a spectrum of phenotypes from pro-inflammatory (M1-like) to reparatory (M2-like) behaviors. Tissue repair is often hampered by a prolonged inflammatory response, contributing to chronic inflammation, fibrosis, and organ failure.

FIGS. 2A-2F: Development and characterization of CDNPs. FIG. 2A: Schematic of cyclodextrin nanoparticle (CDNP) preparation through EDC/NHS-mediated crosslinking of succinyl-β-cyclodextrin by L-lysine with subsequent drug loading by guest-host interaction. FIGS. 2B-2D: CDNP diameter dependence on CD concentration (10:1 EDC, 0.5:1 lysine; FIG. 2B), the molar ratio of lysine to succinyl groups (3.3% w/v CD, 10:1 EDC; FIG. 2C), and the molar ratio of EDC to succinyl groups (3.3% w/v CD, 0.5:1 lysine; FIG. 2D). FIGS. 2E-2F: CDNP characterization (3.3% w/v CD, 0.5:1 lysine, and 12.5:1 EDC). FIG. 2E: Number average histogram of particle size. Inset values: z-average diameter and polydispersity index (PDI); mean±SD. FIG. 2F: Corresponding representative scanning electron microscopy images. Scale bar: 200 nm. Inset: higher resolution image of a single CDNP. Scale bar: 50 nm.

FIGS. 3A-3D: Self-assembly of polymer nanoparticle hydrogels is composition dependent. FIG. 3A: Schematic of drug-loaded hydrogel assembly by guest-host interaction. FIG. 3B: Oscillatory time sweeps of individual components (20% w/v CDNP, green and 7.5% w/v Ad-HA, blue) and formed polymer-CDNP hydrogel (1:1.5 ratio of 7.5% w/v Ad-HA: 20% w/v CDNP, purple); storage (G′, circles), and loss (G″, squares) moduli at 1.0 Hz, 1.0% strain. FIG. 3C: Storage and loss moduli of hydrogels formed from 82 kDa Ad-HA (Low, left), 337 kDa Ad-HA (Low, middle), and 82 kDa Ad-HA (High, right) at varying volumetric ratios of Ad-HA:CDNP. Time sweeps were performed at 10.0 Hz, 1.0% strain; mean±SD, n=2. FIG. 3D: provides bar graphs showing that properties of injectable hydrogels can be tuned and that viscoelastic properties increase with increasing concentrations of Ad-HA.

FIGS. 4A-4D: Guest-host assembled polymer-nanoparticle hydrogels are shear-thinning and self-healing for ease of injection. FIG. 4A: Stress (circle) and viscosity (square) of 82 kDa Ad-HA (High) as a function of shear rate. FIG. 4B: Shear-thinning and self-healing characterization of optimally formulated 82 kDa Ad-HA (High) hydrogel (7.5% w/v) under repeated deformation at 1.0% (low, shaded) and 500% (high) strain at 10.0 Hz; G′ (circle), G″ (square). FIG. 4C: Strain amplitude sweep of 82 kDa Ad-HA (High); G′ (circle), G″ (square). FIG. 4D: Representative images of hydrogel injection; 28 G needle, 1 mL syringe.

FIGS. 5A-5B: Initial drug screening identifies pharmacological inhibitors of M1-like MF activation. FIG. 5A: RAW-Blue™ cells were concurrently treated by zymosan (100 μg/mL) and the indicated drug, spanning a dose response from 100 μM to 31.6 nM in half-log titration to identify M1-like inhibitors. FIG. 5B: Heatmap of compound bioactivities in reporter cells. Results represent the mean of n=4 independent samples, subsequent to subtraction of untreated controls and normalization to zymosan-treated cells such that a lighter color indicates desired inhibition of M1-like response. Within each drug class, compound bioactivity is rank ordered according to cumulative suppression of M1-suppression across all doses. Compounds selected for subsequent evaluation are indicated.

FIGS. 6A-6C: Transcriptional analysis of murine macrophage phenotype. FIG. 6A: RAW264.7 cells were concurrently treated by zymosan (100 μg/mL) and the indicated drug (1 μM) to identify M1-like inhibition and M2-like promotion for all datasets presented. FIG. 6B: Heat map of gene expression following 24 hrs treatment, expressed as ΔΔCt relative to hprt and zymosan-treated controls. Compounds selected for further evaluation are boxed in blue (piclamilast) and orange (celastrol). Results represent the mean of n=3, and are normalized to a housekeeping hprt gene, as well as an internal M1-like control. FIG. 6C: Heatmap of nanoString data represented as the row Z-score of log-transformed normalized data. Genes that were not expressed above background are excluded from presentation. Bone Marrow derived macrophages (BMDMs) were subject to celastrol and piclamilast treatment (1 μM) with concurrent stimulation by zymosan (100 μg/mL). M1 and M2 controls are included for reference. Accompanying cluster analysis and dendogram most closely associate celastrol treatment with M2 controls, while piclamilast treatments are intermediate to M1 and M2 control phenotypes.

FIGS. 7A-7B: Response of human macrophages to celastrol treatment. Human monocyte-derived MF were subject to LPS activation (100 ng/mL) and celastrol treatment at varying doses (1-10 μM). The expression of both M1-associated (FIG. 7A) and M2-associated (FIG. 7B) genes is presented as gene counts relative to untreated (MO) controls; M1 controls are included for reference.

FIGS. 8A-8E: Therapeutic nanoparticle erosion enables long-term modulation of MF phenotype. FIG. 8A: Schematic of drug loaded CDNP release from shear-thinning hydrogels and uptake by MF for desired decrease in inflammatory response. FIG. 8B: Binding sensograms between celastrol and CD, assessed at increasing concentrations of celastrol. FIG. 8C: Cumulative erosion for hydrogels formed from 337 kDa Ad-HA (Low, brown), 82 kDa Ad-HA (Low, blue), and 82 kDa Ad-HA (High, purple); mean±SD, n=4. FIG. 8D: RAW264.7 cell uptake of unloaded (CDNP) and drug-loaded (CDNP-Cel) nanoparticles from media conditioned by 82 kDa Ad-HA (High) hydrogel erosion. Representative images (left) show punctate accumulation of CDNP-AF555. Scale bars, 10 μm. Quantification of fluorescence per cell (right), normalized to unloaded CDNP. FIG. 8E: Anti-inflammatory activity of drug release samples, performed in RAW Blue™ cells by concurrent zymosan stimulation (100 μg/mL) and treatment with conditioned media from 82 kDa Ad-HA (High) hydrogels; p value<0.0001 for all samples relative to zymosan-treated controls (dashed line, red). Data represent the mean±SD, n=3.

FIGS. 9A-9B: ¹H-NMR spectra of the tetrabutylammonium salt of 82 kDa (FIG. 9A) and 337 kDa (FIG. 9B) hyaluronic acid (HA-TBA). The ratio of TBA to HA repeat units is determined by the integration of the TBA methyl group (12H, shaded brown) relative to the N-acetyl group of HA (3H, shaded gray).

FIG. 10A: Chemical structure of adamantane functionalized hyaluronic acid (Ad-HA). FIGS. 10B-10D: ¹H-NMR spectra of Ad-HA products. The degree of substitution (DS) is determined by integration of the ethyl multiplet of adamantane (12H, shaded blue) relative to the sugar ring of HA (10H, shaded gray). For low molecular weight HA (82 kDa), a DS of 18% (FIG. 10B) and 43% (C) yield an average of 37 and 88 Ad per HA macromer, respectively. FIG. 10D: for high molecular weight HA (337 kDa), a DS of 10% afforded a calculated average of 85 guest groups per macromer.

FIG. 11: Temporal dependence of rheological properties of hydrogels composed of 82 kDa Ad-HA (Low), 1:1 ratio Ad-HA:CDNP. Time sweeps were performed at 10.0 Hz, 1.0% strain; G′ (dark blue), G″ (light blue); mean±SD, n=2.

FIGS. 12A-12C: Storage (G′, darker shading) and loss (G″, lighter shading) moduli of 82 kDa Ad-HA (Low), 1:1 ratio Ad-HA:CDNP (FIG. 12A), 337 kDa Ad-HA (Low), 1:0.5 ratio Ad-HA:CDNP (FIG. 12B), and 82 kDa Ad-HA (High), 1:1.5 ratio Ad-HA:CDNP (FIG. 12C) dependent on the concentration of Ad-HA. Time sweeps were performed at 10.0 Hz, 1.0% strain; mean±SD, n=2.

FIG. 13: Representative images of shear-thinning hydrogel injection through a 21 G needle fitted to a 1 mL syringe.

FIG. 14: Cytotoxicity assessment of drug compounds. RAW264.7 cells were treated with indicated drug, spanning a dose-response from 100 μM to 31.6 nM in half-log titration. Heat map represents cell viability, as determined by PrestoBlue™ following normalization to untreated control wells. Data represent the mean of n=3 independent samples.

FIGS. 15A-15B: Expression of M1-associated (FIG. 15A) and M2-associated (FIG. 15B) genes following treatment by celastrol (orange) or piclamilast (blue) in BMDMs. M2 controls (green) are included for reference; p<0.05 for all comparisons unless indicated (ns=not significant). Results represent the group Z-score relative to M1-like controls; mean±SD, n=3.

FIG. 16: Heatmap of expressed genes represented as the row Z-score of log-transformed normalized data. Genes that were not expressed above background are excluded from presentation. Human monocytederived MF were subject to celastrol treatment at varying doses concurrent with LPS activation (100 ng/mL). M1 controls are included for reference. Low (1, 2 μM) doses clustered similarly, while higher doses (5, 10 μM) further diverged from M1 transcriptional profiles.

FIGS. 17A-17B: Hydrogel erosion is dependent on polymer concentration and the ratio of guest-to-host components. Hydrogel erosion occurred over a period of greater than one month, with minimal dependence on polymer concentration (FIG. 17A; 82 kDa Ad-HA (Low); 2.5-10% w/v) and a more pronounced dependence on the volumetric ratio of Ad-HA-to-CDNP (FIG. 17B; 82 kDa Ad-HA (High); 1:0.25-1:4 ratio). Data represent the mean±SD, n=4.

FIG. 18: Cell viability in response to guest and host hydrogel components. Cells were assessed by PrestoBlue™ after 24 h of exposure to the individual components (CDNP or 82 kDa Ad-HA (High)) dissolved in media at varying concentrations. Data represent the mean±SD, n=3, ****p value<0.0001 relative to untreated controls.

FIG. 19A-19C: Representative oscillatory frequency sweeps of drug loaded (FIG. 19A, closed symbol) and unloaded (FIG. 19B, open symbol) hydrogels, and overlaid response (FIG. 19C) for 82 kDa Ad-HA (High) at a 1:1.5 Ad-HA:CDNP ratio; G′ (dark purple), G″ (light purple).

FIGS. 20A-20B: Anti-inflammatory activity of drug release samples performed in RAW-Blue™ cells by concurrent zymosan stimulation (100 μg/mL) and treatment with conditioned media from 82 kDa Ad-HA (FIG. 20A; Low) and 337 kDa Ad-HA (FIG. 20B; Low) hydrogels; p value<0.0001 for all samples relative to zymosan treated controls (dashed line, red). Data represent the mean±SD, n=3.

FIGS. 21A-21B: Anti-inflammatory activity of drug release samples performed in RAW-Blue™ cells by concurrent zymosan stimulation (100 μg/mL) and treatment with conditioned media from 82 kDa Ad-HA (FIG. 21A; Low) hydrogels at varying concentrations of Ad-HA and 82 kDa Ad-HA (FIG. 21B; High) hydrogels at varying ratios of Ad-HA:CDNP; p value<0.0001 for all samples relative to zymosan-treated controls (dashed line, red). Data represent the mean±SD, n=3.

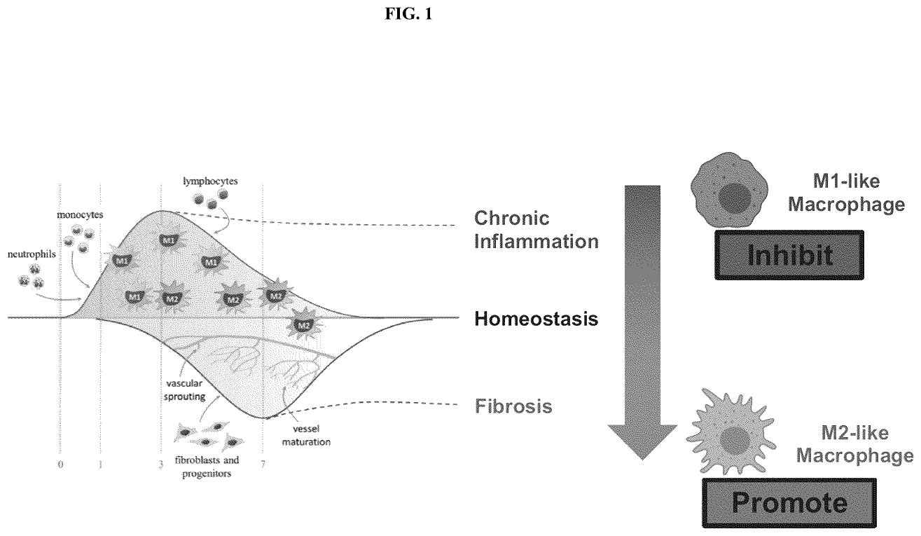

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Hydrogels are conventionally formed ex vivo via covalent crosslinking, necessitating invasive implantation of the resulting solid material. In contrast, injectable hydrogels may be formed through varying methods to enable minimally invasive delivery, including for the local presentation of encapsulated therapeutic cargo. One such approach is in situ hydrogel formation after the injection of liquid precursors, accomplished by mechanisms such as radical polymerization, addition crosslinking, or environmental stimuli. However, the kinetics of hydrogel formation often complicate the delivery process. Rapid gelation risks clogging the delivery device, whereas slow gelation results in material dispersion throughout the tissue after injection.

As an alternative to in situ hydrogel formation, some approaches capitalize on the use of dynamic crosslinks, such as dynamic covalent chemistries, engineered biomolecular interactions, or guest-host chemistries. Dynamic crosslinking allows hydrogels to be formed ex vivo and then extruded during injection by shear-thinning processes, as the dynamic bonds temporarily break in response to shear stress and rapidly bind again. These dynamic material behaviors ultimately allow for injectable delivery that mitigates the risks for invasive implantation, while also increasing material retention at the target site, including precious therapeutic cargo.

As an extension of these self-assembling hydrogel systems, hydrogels may also be formed from discrete micro- or nano-structural units instead of polymeric building blocks alone. Such non-homogenous structures uniquely allow for the structural subunits to take on discrete functions and the unique behavior of the bulk hydrogel formed. For example, granular hydrogels have emerged as a unique class of injectable materials, assembled through inter-particle crosslinking reactions or particle jamming. Discrete microgel components within these systems may have tunable behavior to control drug delivery. Other systems have leveraged polymer-nanoparticle interactions to aid in the self-assembly of injectable hydrogels. The inclusion of discrete nanoparticles within these structures has been leveraged to endow polymer-nanoparticle hydrogels with distinct therapeutic, diagnostic, and physical properties. While such polymer-nanoparticle hydrogels open new avenues for local therapeutic delivery, their use as a means to provide controlled release of nanoparticles to naturally target highly phagocytic innate immune cells remains unexplored.

Such local and cell-specific delivery is particularly important for immune modulation. Specifically, the systemic administration of immunosuppressive drugs places patients at an increased risk of infection, which has hampered their clinical acceptance. Despite these challenges, there remains a growing interest in the use of immunomodulatory drugs to combat inflammatory disease and promote tissue healing.

Macrophages (MF) are crucial regulators of the tissue-immune microenvironment, and unsolicited MF-derived inflammation commonly underlies failed tissue healing processes and an array of chronic diseases, making them an attractive therapeutic target. While these cells exist across an array of phenotypes, they are often broadly characterized as pro-inflammatory (‘M1-like’) or pro-healing (‘M2-like’), particularly in the context of tissue repair. MF phenotypes are highly plastic, subject to modulation by external stimuli such as pharmaceuticals that arrest inflammatory behavior and promote a reparative M2-like phenotype for injury resolution. While immunosuppressants are widely available and used in clinical practice, few M2-polarizing drugs have been reported and necessitate high dosing. Immunoregenerative medicine therefore remains limited by a lack of knowledge regarding which drugs or drug classes can potently promote reparatory MF phenotypes.

Small molecule drugs may be of use towards the goal of modulating MF phenotype and are amenable to delivery by guest-host interactions. Guest-host interactions are a subset of supramolecular associations, characterized by the transient complexation of a macrocyclic host with a small molecule guest. In the case of many macrocycles, and β-cyclodextrin (CD) in particular, complexation is driven by hydrophobic interactions that enable the inclusion of a wide variety of guest molecules. It is therefore a versatile and common excipient in pharmaceutical formulations on market, used to enhance drug solubility and bioavailability. CD is also widely used in biomaterial applications, including in molecular imaging probes, surface coatings, and polymeric drug carriers for affinity-based delivery.

The development of cyclodextrin nanoparticles (CDNPs) that possess a high drug loading capacity and inherent capacity for MF-targeted therapy have been previously described in the literature (U.S. Patent Publication No. US2020/0338011, which is hereby incorporated herein by reference in its entirety). The CDNPs are formed only from CD crosslinked by L-lysine, creating a dense network of the host macrocycle that perpetuates a high drug loading capacity. Due to the saccharide-based structure of CD, it is readily internalized by MF, likely via recognition by cell surface receptors that include scavenger receptor A1 (SR-A1) and mannose receptor (MRC1). The drug loading capacity and MF avidity have been leveraged for systemically administered MF-targeted cancer immunotherapy, and similar MF-targeting strategies are highly effective in a range of applications. However, the delivery of these and other therapeutic nanocarriers is typically accomplished via systemic administration; their local and sustained delivery has not previously been reported.

In one aspect, the disclosure relates to the development of an injectable hydrogel platform for the local administration of small molecule drugs that uniquely leverages guest-host interactions for injectable hydrogel assembly and cell-targeted nanoparticle therapy to directly address the need for location-specific modulation of MF behavior. Through supramolecular assembly by guest-host interactions, CDNPs were crosslinked with adamantane-modified hyaluronic acid (Ad-HA) to yield an injectable hydrogel. The same guest-host interactions also serve as a mechanism to retain immunomodulatory therapeutics. The drug of choice was selected by scrutiny of a targeted library of 45 small molecules drugs that spanned a variety of drug classes with reported immunomodulatory capacity. Through a two-step drug screening process and subsequent in-depth transcriptional analysis of both human and murine MF, celastrol was identified as a potent modulator of MF phenotype that suppresses M1-like and promotes M2-like behavior. Celastrol was included within the nanoparticle core by guest-host interaction prior to hydrogel formation. The resulting therapeutic hydrogels degraded by surface erosion over the course of greater than one month, continually releasing drug-loaded nanoparticles that were uptaken by MF and arrested their pro-inflammatory response. The approach represents a promising strategy to achieve functional re-orientation of the local immune microenvironment by the continual release of drug-loaded nanoparticles from the hierarchical hydrogels formed.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C≡C(CH₃), —C═C(CH₂CH₃), —CH₂C═CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

A disease or disorder is “ameliorated” if the severity of a symptom of the disease or disorder, the frequency with which such a symptom is experienced by a patient, or both, is reduced.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof. Use of the term “aryl” in combination with another term (e.g., iodide, chloride, boronic acid, and magnesium halide, inter alia), indicates that the aryl group is substituted at one or more positions with the substituent defined by the term used in the combination. For example, an aryl chloride indicates that the aryl is substituted with at least one chloride.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical

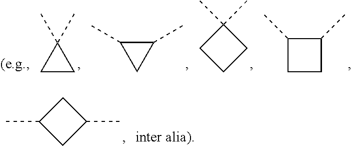

In certain embodiments, the term may be regarded as a product of removal of two hydrogen atoms from the corresponding cycloalkane (e.g., cyclobutyl) by removal of two hydrogen atoms from the same

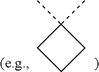

different

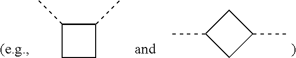

carbon atoms.

A “disease” is a state of health of an animal wherein the animal cannot maintain homeostasis, and wherein if the disease is not ameliorated then the animal's health continues to deteriorate.

As used herein, the term “derived from” refers to a compound or moiety that is structurally identical in most respects to the compound to which it refers. In some embodiments, the compound that the moiety is derived from was used as a reagent or intermediate in the synthesis of the compound that is substituted with the moiety. In some embodiments, the moiety only differs structurally from the compound it is derived from at the portion of the moiety that links to the remainder of the molecule that the moiety substitutes. As used herein, a “derivative” of a particular compound or moiety encompasses compounds and moieties that are derived from the particular compound. For example, 2-hydroxypropyl-α-cyclodextrin is derived from α-cyclodextrin.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

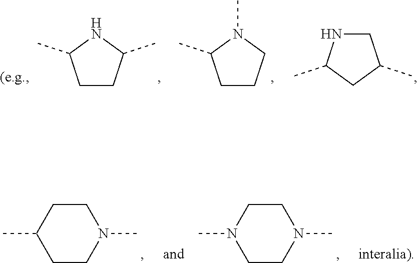

In certain embodiments, the term may be regarded as a product of removal of two hydrogen atoms from the corresponding heterocycloalkane (e.g., piperidine) by removal of two hydrogen atoms from the same

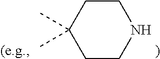

different

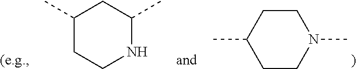

carbon atom(s) and/or heteroatom(s).

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “linker” as used herein refers to an organic moiety that connects two parts of a compound (e.g., two macrocycles). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use herein and methods of preparation thereof.

As used herein, the term “macrocycle” refers to any compound or chemical group that is a cyclic group comprising a minimum of 12 ring members (e.g., 12 or more contiguous atoms that form a ring), wherein the cyclic group is capable of binding a compound (e.g., a therapeutic agent, e.g., an anticancer agent) by means of intermolecular forces that, under certain conditions, last greater than 1 second (e.g., greater than 2 seconds, 4 seconds, 10 seconds, 60 seconds 1 minute, 2 minutes, 5 minutes, 20 minutes, 30 minutes, 1 hour, 2 hours, 4 hours, 1 day, 3 days, 1 week, 2 weeks, 1 month, 2 months, 6 months, 1 year, 2 years, 5 years, or 10 years). Example classes of host macrocycles include cyclodextrins, pillar[n]arenes, calix[n]arenes, and cucurbit[n]urils. Included in each of the foregoing classes are macrocycles derived from any members of that class through, for example, chemical derivatization. For example, “cyclodextrin” encompasses α-cyclodextrin, β-cyclodextrin, and γ-cyclodextrin, as well as any chemically derivatized versions of the same including, but not limited to, 2-hydroxypropyl-α-cyclodextrin, 2-hydroxypropyl-β-cyclodextrin 2-hydroxypropyl-7-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

The term “polymer” may include, according to some embodiments, any molecule comprising repeating structural units connected to each other, typically, by covalent chemical bonds. The term “polymer” may include, according to some embodiments, a homopolymer (which is a polymer derived from one monomer species), a copolymer (which is a polymer derived from two (or more) monomeric species) or a combination thereof. A polymer, as referred to herein, may include a mixture of polymers. A polymer, as referred to herein, may include linear and/branched polymers which consist of a single main chain with one or more polymeric side chains.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Hydrogel Compositions

In one aspect, the disclosure provides a hydrogel composition. In certain embodiments, the hydrogel composition comprises a nanoparticle comprising a plurality of macrocycles, wherein each of the macrocycles is independently covalently linked, either directly or indirectly, by at least one independently selected nanoparticle linker. In certain embodiments, the hydrogel composition comprises a functionalized polymer comprising a hydrophilic polymer core substituted with a plurality of independently selected hydrophobic substituents, wherein at least a portion of the hydrophobic substituents are independently non-covalently associated with one of the macrocycles of the nanoparticle. In certain embodiments, the hydrogel composition comprises at least one therapeutic agent, wherein the at least one therapeutic agent is non-covalently associated with a macrocycle of the nanoparticle.

In certain embodiments, the nanoparticle linker comprises a moiety of Formula (I):

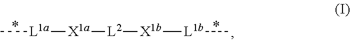

wherein:
L^{1a} and L^{1b} each independently comprise at least one moiety selected from the group consisting of —C(═O)—, -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
L² comprises at least one moiety selected from the group consisting of -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
X^{1a} and X^{1b} are each independently selected from the group consisting of —N(R′)—, —O—, and —S—;
each occurrence of R¹ is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl;
each bond [symbol] comprises a bond between the nanoparticle linker and the macrocycle; and
each occurrence of optionally substituted alkyl, optionally substituted alkylenyl, optionally substituted cycloalkyl, optionally substituted cycloalkylenyl, optionally substituted heteroalkyl, optionally substituted heteroalkylenyl, optionally substituted heterocycloalkyl, optionally substituted heterocycloalkylenyl, optionally substituted aryl, optionally substituted arylenyl, optionally substituted heteroaryl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In certain embodiments, L^{1a} comprises —C(═O)—. In certain embodiments L^{1a} comprises —(CH₂)—. In certain embodiments, L^{1a} comprises —(CH₂)₂—. In certain embodiments L^{1a} comprises —(CH₂)—. In certain embodiments, L^{1a} comprises —(CH₂)₃—. In certain embodiments L^{1a} comprises —(CH₂)—. In certain embodiments, L^{1a} comprises —(CH₂)₄—. In certain embodiments, L^{1a} comprises

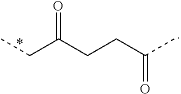

In certain embodiments, L^{1b} comprises —C(═O)—. In certain embodiments L^{1b} comprises —(CH₂)—. In certain embodiments, L^{1b} comprises —(CH₂)₂—. In certain embodiments L^{1b} comprises —(CH₂)—. In certain embodiments, L^{1b} comprises —(CH₂)₃—. In certain embodiments L^{1b} comprises —(CH₂)—. In certain embodiments, L^{1b} comprises —(CH₂)₄—. In certain embodiments, L^{1b} is

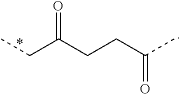

In certain embodiments, X^{1a} is —NH—. In certain embodiments, X^{1b} is —NH—.

In certain embodiments, L² comprises —[CH(C(═O)OH)]—. In certain embodiments L² comprises —(CH₂)—. In certain embodiments, L² comprises —(CH₂)₂—. In certain embodiments L² comprises —(CH₂)—. In certain embodiments, L² comprises —(CH₂)₃—. In certain embodiments L² comprises —(CH₂)—. In certain embodiments, L² comprises —(CH₂)₄—. In certain embodiments, L² is

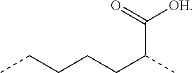

In certain embodiments, the nanoparticle linker is

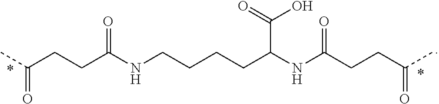

In certain embodiments, at least one macrocycle is a cyclodextrin. In certain embodiments, at least one macrocycle is a pillar[n]arene. In certain embodiments, at least one macrocycle is a calix[n]arene. In certain embodiments, at least one macrocycle is a cucurbit[n]uril.

In certain embodiments, the cyclodextrin is an α-cyclodextrin. In certain embodiments, the cyclodextrin is a β-cyclodextrin. In certain embodiments, the cyclodextrin is a 7-cyclodextrin.

In certain embodiments, the cyclodextrin is a 2-hydroxypropyl-α-cyclodextrin. In certain embodiments, the cyclodextrin is a 2-hydroxypropyl-β-cyclodextrin. In certain embodiments, the cyclodextrin is a 2-hydroxypropyl-7-cyclodextrin. In certain embodiments, the cyclodextrin is a methyl-α-cyclodextrin. In certain embodiments, the cyclodextrin is a methyl-β-cyclodextrin. In certain embodiments, the cyclodextrin is a methyl-γ-cyclodextrin. In certain embodiments, the cyclodextrin is a cyclodextrin sulfobutylether. In certain embodiments, the cyclodextrin is a cyclodextrin thioether. In certain embodiments, the cyclodextrin is a cyanoethylated cyclodextrin. In certain embodiments, the cyclodextrin is a succinyl-cyclodextrin. In certain embodiments, the cyclodextrin is an aminated cyclodextrin.

In certain embodiments, the nanoparticle linker is covalently conjugated to a primary hydroxyl of the cyclodextrin.

In certain embodiments, the hydrophilic polymer core is a hyaluronic acid derivative. In certain embodiments, the hydrophilic polymer core is a dextran derivative. In certain embodiments, the hydrophilic polymer core is a chitosan derivative. In certain embodiments, the hydrophilic polymer core is a fucoidan derivative. In certain embodiments, the hydrophilic polymer core is an alginate derivative. In certain embodiments, the hydrophilic polymer core is a cellulose derivative. In certain embodiments, the hydrophilic polymer core is a collagen derivative. In certain embodiments, the hydrophilic polymer core is a poly(ethylene glycol) derivative. In certain embodiments, the hydrophilic polymer core is a poly(hydroxyethyl acrylate) derivative. In certain embodiments, the hydrophilic polymer core is a poly(hydroxyethyl methacrylate) derivative. In certain embodiments, the hydrophilic polymer core is a poly(N-isopropylacrylamide) derivative. In certain embodiments, the hydrophilic polymer core is a poly(glycolic acid). In certain embodiments, the hydrophilic polymer core is a poly(lactic acid) derivative. In certain embodiments, the hydrophilic polymer core is a poly(lactic acid-glycolic acid) derivative. In certain embodiments, the hydrophilic polymer core is an oligo(poly(ethylene glycol)fumarate) derivative. In certain embodiments, the hydrophilic polymer core is a poly(vinyl alcohol) derivative. In certain embodiments, the hydrophilic polymer core is a poly(vinyl acid) derivative.

In certain embodiments, the hydrophobic substituent comprises a linear, branched, cyclic, or polycyclic C₆-C₂₀ hydrocarbon, C₆-C₂₀ aryl or alkylaryl, hetero or alkylaromatic hydrocarbon moieties. Additional hydrophobic substituents contemplated for use herein are described in the literature, for example U.S. Pat. No. 9,827,321, which is hereby incorporated herein by reference in its entirety. See also Bioconjugate Chem. 2015, 26, 2279-2289; and Coord. 2022, 454:214352.

In certain embodiments, the macrocycle comprises an α-cyclodextrin and the hydrophobic substituent comprises hexyl. In certain embodiments, the macrocycle comprises an α-cyclodextrin and the hydrophobic substituent comprises octyl. In certain embodiments, the macrocycle comprises an α-cyclodextrin and the hydrophobic substituent comprises polyethylene oxide. In certain embodiments, the macrocycle comprises an α-cyclodextrin and the hydrophobic substituent comprises ferrocenyl. In certain embodiments, the macrocycle comprises an α-cyclodextrin and the hydrophobic substituent comprises azobenzyl.

In certain embodiments, the macrocycle comprises a β-cyclodextrin and the hydrophobic substituent comprises adamantyl. In certain embodiments, the macrocycle comprises a 3-cyclodextrin and the hydrophobic substituent comprises cyclohexyl. In certain embodiments, the macrocycle comprises a β-cyclodextrin and the hydrophobic substituent comprises benzyl. In certain embodiments, the macrocycle comprises an β-cyclodextrin and the hydrophobic substituent comprises ferrocenyl. In certain embodiments, the macrocycle comprises an β-cyclodextrin and the hydrophobic substituent comprises azobenzyl.

In certain embodiments, the macrocycle comprises a γ-cyclodextrin and the hydrophobic substituent comprises cyclodecyl. In certain embodiments, the macrocycle comprises an γ-cyclodextrin and the hydrophobic substituent comprises ferrocenyl. In certain embodiments, the macrocycle comprises an γ-cyclodextrin and the hydrophobic substituent comprises azobenzyl.

In certain embodiments, the macrocycle comprises a cucurbit[6]uril and the hydrophobic moiety comprises a polyamine. In certain embodiments, the polyamine is a hexanediamine. In certain embodiments, the polyamine is spermine.

In certain embodiments, the hydrophobic substituent is independently adamantyl.

In certain embodiments, the functionalized polymer is a compound of Formula (II):

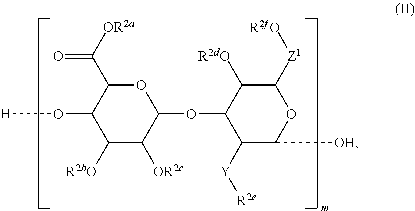

wherein:
each occurrence of R^{2a}, R^{2b}, R^{2c}, R^{2d}, R^{2e}, and R^{2f} is independently selected from the group consisting of R³, H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl,
   wherein the compound of Formula (II) comprises n occurrences of R³,
   wherein 0<n≤m, and
   wherein no more than one of R^{2a}, R^{2b}, R^{2c}, R^{2d}, R^{2e}, and R^{2f} in each repeated unit of the compound Formula (II) is R³;
R³ is

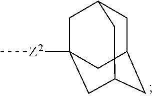

Y is selected from the group consisting of —N(R^{I})— and —O—;
Z¹ and Z² are each independently selected from the group consisting of -(optionally substituted C₁-C₆ alkylenyl)-, -(optionally substituted C₁-C₆ heteroalkylenyl)-, —C(═O)(optionally substituted C₁-C₆ alkylenyl)-, —C(═O)(optionally substituted C₁-C₆ heteroalkylenyl)-, -(optionally substituted C₁-C₆ alkylenyl)C(═O)—, and -(optionally substituted C₁-C₆ heteroalkylenyl)C(═O)—;
m is an integer ranging from 2 to 1000;
n is an integer ranging from 1 to 999; and
each occurrence of R^{I} and R^{II} is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl.

In certain embodiments, the compound of Formula (II) is a compound of Formula (IIa):

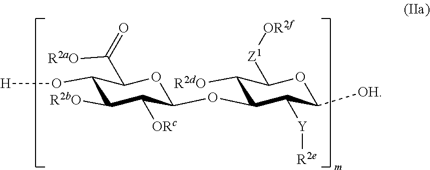

In certain embodiments, each occurrence of R^{2a}, R^{2b}, R^{2c}, R^{2d}, and R^{2e} is independently selected from the group consisting of H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl.

In certain embodiments, R^{2f} is H. In certain embodiments, R^{2f} is R³.

In certain embodiments, R^{2a} is H. In certain embodiments, R^{2b} is H. In certain embodiments, R^{2c} is H. In certain embodiments, R^{2d} is H. In certain embodiments, R^{2e} is H. In certain embodiments, R^{2e} is C(═O)Me.

In certain embodiments, Y is —NH—.

In certain embodiments, Z¹ is —CH₂—.

In certain embodiments, Z² is —C(═O)CH₂—.

In certain embodiments, R³ is

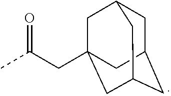

In certain embodiments, m is an integer selected from the group consisting of about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000.

In certain embodiments, n and m have a ratio of about 1:10, 1:9, 1:8, 1:7, 1:6, 1:5, 1:4, 1:3, or about 1:2.

In certain embodiments, the therapeutic agent is a small molecule therapeutic agent.

In certain embodiments, the therapeutic agent is an immunomodulatory and/or anti-inflammatory agent.

In certain embodiments, the anti-inflammatory agent is at least one selected from the group consisting of NSAIDs, steroids, glucocorticoids, statins, PPAR inhibitors, PDE4 inhibitors, resolvins, antibiotics, quinones, ROS-scavengers, or naturally-derived compounds, and the like.

In certain embodiments, the anti-inflammatory agent is celastrol. In certain embodiments, the anti-inflammatory agent is piclamilast. In certain embodiments, the anti-inflammatory agent is ciglitazone. In certain embodiments, the anti-inflammatory agent is celecoxib. In certain embodiments, the anti-inflammatory agent is betamethasone. In certain embodiments, the anti-inflammatory agent is pravastatin. In certain embodiments, the anti-inflammatory agent is aspirin. In certain embodiments, the anti-inflammatory agent is ketorolac. In certain embodiments, the anti-inflammatory agent is lornoxicam. In certain embodiments, the anti-inflammatory agent is cortisone. In certain embodiments, the anti-inflammatory agent is pioglitazone. In certain embodiments, the anti-inflammatory agent is prednisolone. In certain embodiments, the anti-inflammatory agent is triamcinolone. In certain embodiments, the anti-inflammatory agent is methylprednisolone. In certain embodiments, the anti-inflammatory agent is desoximetasone. In certain embodiments, the anti-inflammatory agent is dexamethasone. In certain embodiments, the anti-inflammatory agent is fluvastatin. In certain embodiments, the anti-inflammatory agent is lovastatin. In certain embodiments, the anti-inflammatory agent is simvastatin. In certain embodiments, the anti-inflammatory agent is atorvastatin. In certain embodiments, the anti-inflammatory agent is rosuvastatin. In certain embodiments, the anti-inflammatory agent is gemfibrozil. In certain embodiments, the anti-inflammatory agent is troglitazone. In certain embodiments, the anti-inflammatory agent is rolipram. In certain embodiments, the anti-inflammatory agent is fenofibrate. In certain embodiments, the anti-inflammatory agent is rosiglitazone. In certain embodiments, the anti-inflammatory agent is apremilast. In certain embodiments, the anti-inflammatory agent is cilomilast. In certain embodiments, the anti-inflammatory agent is crisoborole. In certain embodiments, the anti-inflammatory agent is roflumilast. In certain embodiments, the anti-inflammatory agent is iloprost. In certain embodiments, the anti-inflammatory agent is colchicine. In certain embodiments, the anti-inflammatory agent is quercetin. In certain embodiments, the anti-inflammatory agent is JSH-23. In certain embodiments, the anti-inflammatory agent is tuftsin. In certain embodiments, the anti-inflammatory agent is resolving D1. In certain embodiments, the anti-inflammatory agent is sivelestat. In certain embodiments, the anti-inflammatory agent is doxycycline. In certain embodiments, the anti-inflammatory agent is methotrexate. In certain embodiments, the anti-inflammatory agent is vortioxetine.

In certain embodiments, the therapeutic agent is a pro-inflammatory agent.

In certain embodiments, the pro-inflammatory agent is at least one selected from the group consisting of tyrosine kinase inhibitors, CSF1R inhibitors, mTOR inhibitors, or direct immune agonists that include agonists of toll-like receptors, NOD-like receptors, STING, or other pattern-recognition receptors, and the like.

In certain embodiments, the pro-inflammatory agent is GW2580. In certain embodiments, the pro-inflammatory agent is CEP32496. In certain embodiments, the pro-inflammatory agent is BLZ945. In certain embodiments, the pro-inflammatory agent is OSI930. In certain embodiments, the pro-inflammatory agent is PLX3397. In certain embodiments, the pro-inflammatory agent is dasatinib. In certain embodiments, the pro-inflammatory agent is sunitinib. In certain embodiments, the pro-inflammatory agent is ABT869. In certain embodiments, the pro-inflammatory agent is imatinib. In certain embodiments, the pro-inflammatory agent is foretinib. In certain embodiments, the pro-inflammatory agent is XL228. In certain embodiments, the pro-inflammatory agent is gefitinib. In certain embodiments, the pro-inflammatory agent is PD0325901. In certain embodiments, the pro-inflammatory agent is trametinib. In certain embodiments, the pro-inflammatory agent is bentamapimod. In certain embodiments, the pro-inflammatory agent is dabrafenib. In certain embodiments, the pro-inflammatory agent is vemurafenib. In certain embodiments, the pro-inflammatory agent is crizotinib. In certain embodiments, the pro-inflammatory agent is UNC2025. In certain embodiments, the pro-inflammatory agent is indoximod. In certain embodiments, the pro-inflammatory agent is celecoxib. In certain embodiments, the pro-inflammatory agent is rapamycin. In certain embodiments, the pro-inflammatory agent is NIK12192. In certain embodiments, the pro-inflammatory agent is trichostatin A. In certain embodiments, the pro-inflammatory agent is IBET151. In certain embodiments, the pro-inflammatory agent is TMP195. In certain embodiments, the pro-inflammatory agent is BYL719. In certain embodiments, the pro-inflammatory agent is GDC0941. In certain embodiments, the pro-inflammatory agent is BKM120. In certain embodiments, the pro-inflammatory agent is imiquimod. In certain embodiments, the pro-inflammatory agent is gardiquimod. In certain embodiments, the pro-inflammatory agent is resiquimod (R848). In certain embodiments, the pro-inflammatory agent is motolimod. In certain embodiments, the pro-inflammatory agent is GS9620.

In certain embodiments, the therapeutic agent is at least one selected from the group consisting of JAK inhibitors, sphingosine-1-phosphate receptor modulators, cannabinoid receptor ligands, COX inhibitors, ACE inhibitors, Chalcone derivatives, Indole derivatives, Pyrazole derivatives, Pyrazolo[3,4-d]pyrimidine derivatives, Thiazolidinone derivatives, Triazol derivatives, Oxazolidinone derivatives, Imidazole derivatives, Pyrrol derivatives, or Furan derivatives and the like.

In certain embodiments, the nanoparticle has a diameter of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nm. In certain embodiments, the nanoparticle has a diameter of about 80 nm.

In certain embodiments, the nanoparticle comprises β-cyclodextrin succinate crosslinked with lysine. In certain embodiments, the β-cyclodextrin succinate and lysine have a ratio of about 1:1.

In certain embodiments, the functionalized polymer has a degree of monomer functionalization ranging from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50%. In certain embodiments, the functionalized polymer has a degree of monomer functionalization of about 10%. In certain embodiments, the functionalized polymer has a degree of monomer functionalization of about 18%. In certain embodiments, the functionalized polymer has a degree of monomer functionalization of about 43%. In certain embodiments, the term “degree of functionalization” refers to the degree to which each monomer of the functionalized polymer is substituted with a hydrophobic moiety. For example, in certain embodiments, wherein the hydrophobic moiety is adamantyl and the hydrophilic polymer core is hyaluronic acid, the “degree of functionalization” refers to the amount of disaccharide monomers which are adamantyl-substituted as a percentage of the total number of disaccharide monomers in the hyaluronic acid polysaccharide.

In certain embodiments, the functionalized polymer comprises about 1, 2, 3, 4, 5, 6, 7, 8, 9, 10, 11, 12, 13, 14, or about 15% (w/v) of the hydrogel composition. In certain embodiments, the functionalized polymer comprises about 2.5% (w/v) of the hydrogel composition. In certain embodiments, the functionalized polymer comprises about 5.0% (w/v) of the hydrogel composition. In certain embodiments, the functionalized polymer comprises about 7.5% (w/v) of the hydrogel composition. In certain embodiments, the functionalized polymer comprises about about 10.0% (w/v) of the hydrogel composition.

In certain embodiments, the nanoparticle and the functionalized polymer have a ratio in the hydrogel composition of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or about 1:10 (nanoparticle:functionalized polymer). In certain embodiments, the nanoparticle and the functionalized polymer have a ratio in the hydrogel composition of about 0.5:1.0 (nanoparticle:functionalized polymer). In certain embodiments, the nanoparticle and the functionalized polymer have a ratio in the hydrogel composition of about 1:0:1.0 (nanoparticle:functionalized polymer). In certain embodiments, the nanoparticle and the functionalized polymer have a ratio in the hydrogel composition of about 1.5:1.0 (nanoparticle:functionalized polymer).

In certain embodiments, the therapeutic agent has a concentration in the hydrogel composition of about 5 μM.

### Methods

In another aspect, the disclosure provides a method for treating, preventing, and/or ameliorating a disease or disorder in a subject. In certain embodiments, the method comprises administering to the subject the hydrogel composition of the disclosure.

In certain embodiments, the disease or disorder is an inflammatory disease or disorder. In certain embodiments, the disease or disorder is a focal tissue injury. In certain embodiments, the disease or disorder is an autoimmune disease and/or associated tissue dysfunction. In certain embodiments, the disease or disorder is cancer. In certain embodiments, the disease or disorder is or relates to a cytokine storm. In certain embodiments, the disease or disorder is sepsis.

In certain embodiments, the inflammatory disease or disorder is selected from the group consisting of cardiovascular disease, diabetic wounds, osteoarthritis, inflammatory bowel disease, and colitis. In certain embodiments, the inflammatory disease or disorder is a cardiovascular disease. In certain embodiments, the cardiovascular disease is atherosclerosis. In certain embodiments, the cardiovascular disease is myocarditis. In certain embodiments, the cardiovascular disease is endocarditis.

In certain embodiments, the cardiovascular disease is at least one selected from the group consisting of atherosclerosis, myocarditis, endocarditis, myocardial infarction, heart failure, stroke, aneurism, aortic dissection, peripheral arterial disease, congenital defects, and valvular disease.

In certain embodiments, the focal tissue injury is at least one selected from the group consisting of acute kidney injury, spinal cord injury, traumatic brain injury, muscle loss, and bone fracture

In certain embodiments, the chronic autoimmune disease and associated tissue dysfunction is at least one selected from the group consisting of multiple sclerosis, systemic lupus, rheumatoid arthritis, type 1 or type 2 diabetes, scleroderma, and psoriasis.

In certain embodiments, the cancer is at least one selected from the group consisting of hematologic cancer, breast cancer, lung cancer, head and neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, and skin cancer.

In certain embodiments, the hydrogel composition is locally administered to an inflamed tissue or organ. In certain embodiments, the inflamed tissue or organ is the heart.

In another aspect, the disclosure provides a method for delivering a therapeutic agent to a macrophage in a subject. In certain embodiments, the method comprises administering to the subject the hydrogel composition of the disclosure.

In another aspect, the disclosure provides a method for reducing or inhibiting pro-inflammatory (M1-like) behavior of a macrophage in a subject. In certain embodiments, the method comprises administering to the subject the hydrogel composition of the disclosure.

In another aspect, the disclosure provides a method for promoting pro-healing (M2-like) behavior of a macrophage in a subject. In certain embodiments, the method comprises administering to the subject the hydrogel composition of the disclosure.

In certain embodiments, the subject is a mammal. In certain embodiments, the mammal is a human.

### EXAMPLES

Various embodiments of the present application can be better understood by reference to the following Examples which are offered by way of illustration. The scope of the present application is not limited to the Examples given herein.

### Materials and Methods

### Materials

Unless otherwise indicated, solvents and general reagents were purchased from Sigma-Aldrich or TCI America and used without additional purification. Pharmaceutical drugs were obtained from Selleckchem, MedChemExpress, or Cayman Chemical Company and prepared at stock concentrations of 100 mM in dimethyl sulfoxide (DMSO). Cell culture reagents were purchased from VWR, unless otherwise stated.

### Nanoparticle Synthesis and Characterization

Cyclodextrin nanoparticles (CDNPs) were prepared by methods as previously reported in the literature. Briefly, succinyl-β-cyclodextrin (1.0-3% w/v, 1.0 eq. succinylated groups), 1-ethyl-3-(3-dimethylaminopropyl)carbodiimide (EDC, 5-12.5 eq. to succinylate), and N-hydroxysuccinimide (NHS, 0.5 eq. to EDC) were dissolved in MES buffer (50 mM, pH 6.0). The reaction was stirred (30 min, RT) prior to the dropwise addition of L-lysine (0.5-4 eq. to succinylate) and overnight crosslinking. The product was recovered by addition of 100 μL brine and precipitation from a tenfold excess of anhydrous ethanol on ice. Following immediate re-dissolution in water, the product was purified by size-exclusion chromatography (SEC; PD-10, Fisher). Saccharide positive fractions were identified by spotting on a silica gel TLC plate, developing with 5% H₂SO₄ in ethanol, and then heating the plate. Positive fractions were concentrated by centrifugal filtration (10 kDa MWCO, Amicon), washed repeatedly with water, and lyophilized until dry. The final CDNP products were re-dissolved at 20% w/v in MilliQ water and stored at −20° C. until later use. Particle size was determined by dynamic light scattering (DLS; Zetasizer, Malvern) in triplicate at a concentration of 5 mg/mL in phosphate buffered saline (PBS). For scanning electron microscopy (SEM), samples were prepared at 200 μg/mL in DI water, lyophilized in a thin layer on conductive scaffolding, attached to SEM stubs using double sided carbon tape, and sputter coated prior to imaging (Zeiss, Supra 50VP).

### Polymer Synthesis and Characterization

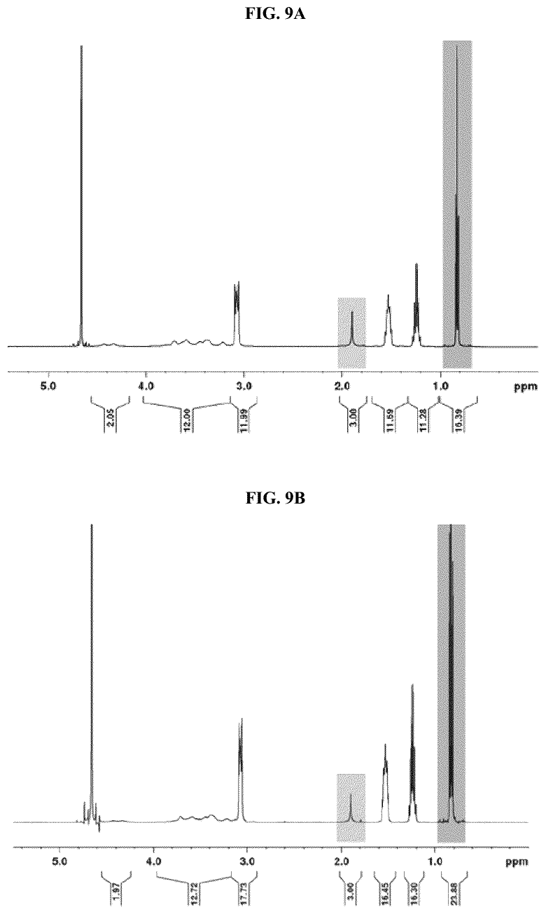

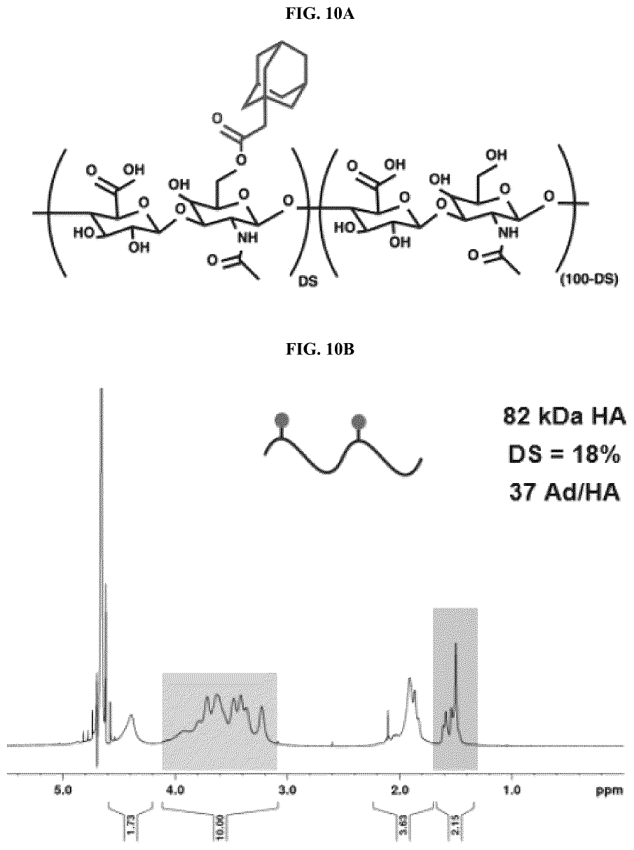

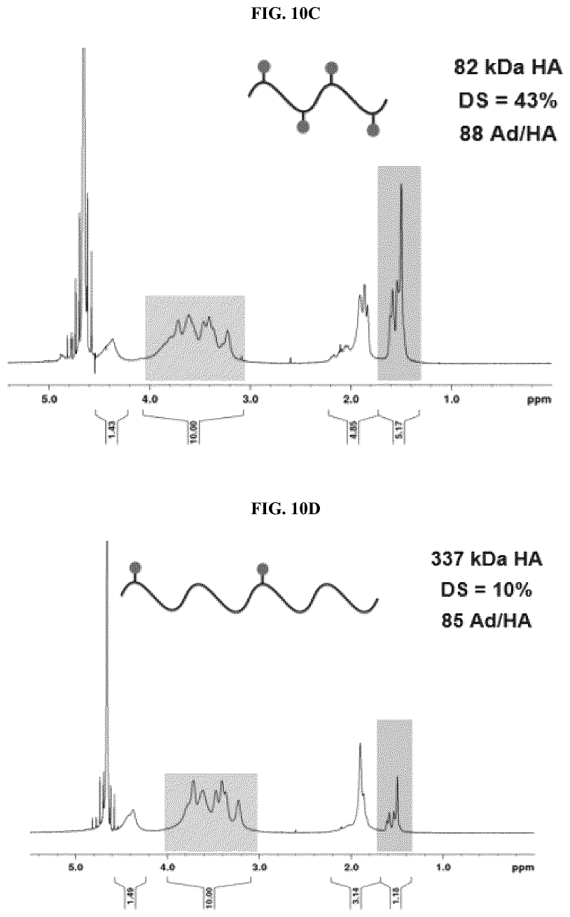

Hyaluronic acid (HA; MW=82 kDa or 337 kDa; Lifecore Biomedical) was modified by pendant addition of 1-adamantane acetic acid (Ad), similar to previous reports. HA (5 g) was dissolved in DI water at 2% w/v, exchanged against Dowex 50W resin (15 g), neutralized by tetrabutylammonium hydroxide (TBA) to a final pH of 7.02-7.05, and lyophilized to yield HA-TBA (FIGS. 9A-9B). Coupling of 1-adamantane acetic acid to HA-TBA proceeded via esterification. A round bottom flask was charged with a stir bar, HA-TBA (1.5 g, 1 mol equiv. HA repeat units), 4-dimethylaminopyridine (DMAP, 0.38 g, 1.5 mol equiv.), Ad (1.2 g, 3 mol equiv.), and blanketed under dry nitrogen. Anhydrous dimethyl sulfoxide (5 mL/0.1 g of HA-TBA) was added via cannulation and reactants dissolved. Di-tert-butyl decarbonate (Boc₂O) was added via syringe (0.23 mL for 82 kDa Ad-HA (Low), 0.20 mL for 337 kDa Ad-HA (Low), and 0.56 mL for 82 kDa Ad-HA (High)) and the reaction allowed to proceed for 20 h at 45° C. The product was dialyzed (8-10 kDa MWCO) against DI water, which was changed twice daily for 14 days. After lyophilization, the degree of substitution of the HA backbone by Ad was determined by ¹H-NMR (FIGS. 10A-10D).

### Hydrogel Formation

Hydrogels were prepared from stock solutions of CDNP (20% w/v) and the denoted Ad-HA polymer (82 kDa Ad-HA (Low), 337 kDa Ad-HA (Low), and 82 kDa Ad-HA (High)) prepared in PBS. Hydrogels were formed by pipette mixing of the two separate solutions, followed by vortexing, manual stirring, and sonication to ensure homogenization with brief centrifugation to remove entrapped air bubbles. Hydrogel formulations were varied by adjusting the initial Ad-HA polymer concentration (2.5-10% w/v), the volumetric ratio of Ad-HA to CDNP (8:1-1:4), and anneal time (0-14 days) following mixing.

### Rheological Characterization

Characterization was performed on a TA Instruments Discovery HR20 rheometer fitted with a cone and plate geometry (20 mm diameter, 1° cone angle, 27 μm gap). Temperature was maintained at 25° C. through use of a Peltier plate stage. Properties of hydrogel samples were examined by oscillatory time sweeps at varying frequencies (0.1, 1, and 10 Hz; 1% strain), oscillatory frequency sweeps (0.01 Hz to 100 Hz; 1% strain), oscillatory strain sweeps (1% to 500% strain; 10 Hz), and continuous flow experiments with the shear rate linearly ramped from 0.005 to 50 s⁻¹. Recovery experiments were performed using oscillatory time sweeps at 500% strain with recovery at 1% strain.

### Cell Culture

Cells were maintained under standard culture conditions (37° C., 5% CO₂) in the indicated medium which was replenished every two days. The murine MF cell lines, RAW 264.7 (ATCC) and RAW-Blue™ (InvivoGen), were cultured in Dulbecco's Modified Eagle Medium (DMEM) supplemented with heat-inactivated fetal bovine serum (FBS), and 1% penicillin-streptomycin (Pen-Strep). RAW-Blue™ media was additionally supplemented by 100 μg/ml Normocin with the addition of 100 μg/ml of Zeocin every other passage to maintain selection pressure, as recommended by the manufacturer. Cells were passaged at 70% confluency. Primary MF were derived from murine and human tissues. Bone marrow derived macrophages (BMDMs) were isolated and derived according to standard protocols. Marrow was extracted from the surgically resected femur and tibia of male C57BL/6 mice, dissociated, and filtered using a 40 μm strainer. Red blood cells were lysed with ammonium chloride, and recovered cells were plated at 2×10⁶ cells/well in 24 well plates and maintained in Iscove's Modification of DMEM (IMDM) supplemented with 10% heat-inactivated FBS, 1% Pen-Strep, and 10 ng/mL recombinant mouse macrophage colony-stimulating factor (M-CSF; PeproTech). Primary human monocytes were isolated from peripheral blood (New York Blood Center) from a single healthy human donor via density centrifugation as previously described in the literature. Harvested monocytes were cultured for 5 days on non-tissue culture-treated well plates in RPMI-1640 media supplemented with 10% heat-inactivated human serum, 1% Pen-Strep, and 20 ng/mL recombinant human M-CSF (PeproTech).

### Reporter Assays and Transcriptional Analysis

The process of drug selection leveraged a unique two-step drug screen that first identified potent M1-inhibitors using a high-throughput reporter cell assay followed by secondary identification of M2-promoting drugs in cell lines. Furthermore, a rigorous approach for validating drug activity was pursued, including in depth transcriptional analysis of primary murine and human cells to ensure cross-species drug activity. For initial drug screens, RAW-Blue™ cells were plated at 1×10⁵ cells/well in 384 well plates. To induce polarization towards an M1-like phenotype, media was replaced after 24 h including zymosan (100 μg/mL). Drug dosing was performed concurrently, spanning concentrations from 100 μM to 0.03 μM in half-log dilutions; n=4 per group. At 24 h after drug treatment, secreted embryonic alkaline phosphatase (SEAP) reporter activity was quantified using QUANTI-Blue™ Solution following the manufacturer's protocol. Absorbance was read at 620 nm (BioTek Instruments, Synergy H1) and is presented following normalization to zymosan-treated controls.

For secondary identification of M2-promoting drugs, transcriptional analysis using qPCR was performed. RAW264.7 cells were plated at 1×10⁶ cells/well in 24 well plates. After 24 h, media was replaced, and IL-4 (10 ng/mL, PeproTech) or zymosan (100 μg/mL) were included to serve as internal controls for M2-like and M1-like phenotypes, respectively. Treatment groups were concurrently activated via zymosan and treated with 1 μM drug concentrations, n=3 per group. After 24 hrs, cells were lysed by freezing, RNA was extracted (RNEasy Mini Kit; Qiagen), and cDNA synthesis was performed (High-Capacity cDNA Reverse Transcription Kit; Fisher). Samples were subject to qPCR using Taqman Fast Advanced Master Mix and probes (Fisher) for analysis of hprt (Mm01545399_m1), nos2 (Mm00440502_m1), il12b (Mm01288989_m1), il6 (Mm00446190_m1), mrc1 (Mm00485148_m1), il10 (Mm01288386_m1), and arg1 (Mm00475988_m1). Data is expressed as a fold change in gene expression using the ΔΔCt method, relative to the hprt and zymosan-treated controls.

For in depth transcriptional profiling of best drug candidates (piclamilast and celastrol), primary murine and human cells were subject to nanoString analysis. BMDMs were differentiated in 24 well plates as described. Zymosan (100 μg/mL) and IL-4 (10 ng/mL) treatments were again included as internal references for M1-like and M2-like MF phenotypes, respectively. Cells were treated with 1 μM of the prescribed compounds for 24 hrs, n=3 per group. Following RNA extraction, nanoString multiplex gene expression analysis was performed using 100 ng of extracted RNA and a custom-designed panel of 91 genes (Table 1), which relate primarily to murine MF phenotypes, as well as angiogenesis and fibrosis. Transcriptional analysis was similarly performed for human MF. Following differentiation from peripheral blood monocytes as described, cells were stimulated with lipopolysaccharide (LPS, 100 ng/mL). After 24 hrs, celastrol was added (1-10 μM, 24 hrs). RNA was isolated (RNAqueous-Micro Total RNA Isolation Kit, Fisher) and nanoString was performed with a custom-designed panel of 70 genes related primarily to MF phenotypes, angiogenesis, and fibrosis (Table 2). For both murine and human datasets, data was normalized to internal positive and negative controls using the nSolver 4.0 software and subsequently normalized to housekeeping genes (geometric mean of hprt and tbp for mouse, gapdh and tbp for human), as recommended by the manufacturer. Data is presented for all genes expressed above background as raw gene counts or as the Z-score of log-transformed data relative to M1-like controls or row means as indicated.

TABLE 1
Murine nanoString gene panel

| Gene | Target Identifier | Annotation | Gene | Target Identifier | Annotation |
|---|---|---|---|---|---|
| Acta2 | NM_007392.2 | ECM | Il1a | NM_010554.4 | M1 |
| Angpt2 | NM_007426.3 | Angiogenesis | Il1b | NM_008361.3 | M1 |
| Arg1 | NM_007482.3 | M2 | Il6 | NM_031168.1 | M1 |
| Axl | NM_009465.4 | M2 | Irf3 | NM_016849.4 | M1 |
| Bbgn | NM_007542.4 | ECM | Irf4 | NM_013674.1 | M2 |
| Birc3 | NM_007464.3 | M1 | Irf5 | NM_001252382.1 | M1 |
| Ccl17 | NM_011332.2 | M2 | Jak1 | NM_146145.2 | Immune |
| | | | | | signaling |
| Ccl2 | NM_011333.3 | M1 | Ki67 | XM_006507413.5 | M1 |
| Ccl22 | NM_009137.2 | M2 | Mer | NM_008587.1 | M2 |
| Ccl4 | NM_013652.1 | M1 | Mmp13 | NM_008607.1 | Protease |
| Ccl5 | NM_013653.1 | M1 | Mmp14 | NM_008608.3 | Protease |
| Ccn2 | NM_010217.2 | ECM | Mmp2 | NM_008610.2 | Protease |
| Cd36 | NM_007643.3 | M1 | Mmp8 | NM_008611.4 | Protease |
| Cd38 | NM_007646.4 | M1 | Mmp9 | NM_013599.2 | Protease |
| Cd68 | NM_009853.1 | M1 | Mrc1 | NM_008625.1 | M2 |
| Cd80 | NM_009855.2 | M1 | Nfkabia | NM_010907.2 | M1 |
| Cd86 | NM_019388.3 | M1 | Nos2 | NM_010927.3 | M1 |
| Chil3 | NM_009892.2 | M2 | Pdgfb | NM_011057.3 | Angiogenesis |
| Col1a1 | NM_007742.3 | ECM | S100a4 | NM_011311.2 | Fibrosis |
| Col6a1 | NM_009933.2 | ECM | Spp1 | NM_009263.3 | ECM |
| Col3a1 | NM_009930.1 | ECM | Stat3 | NM_213659.2 | M2 |
| Csf1r | NM_001037859.1 | Immune | Stat6 | NM_009284.2 | M2 |
| | | signaling | | | |
| Cxcl10 | NM_021274.1 | M1 | Tgfb1 | NM_011577.1 | M2 |
| Cxcl11 | NM_019494.1 | M1 | Timp1 | NM_011593.2 | ECM |
| Cxcl12 | NM_021704.3 | Immune | Timp3 | NM_011595.2 | ECM |
| | | signaling | | | |
| Cxcr4 | NM_009911.3 | M1 | Tnf | NM_013693.2 | M1 |
| Dcn | NM_001190451.1 | ECM | Top2a | NM_011623.2 | M1 |
| Egr2 | NM_010118.2 | M2 | Vcan | NM_172955.1 | ECM |
| Fn1 | NM_010233.1 | ECM | Vegfa | NM_001025250.3 | Angiogenesis |
| H2afx | NM_010436.2 | M1 | Vim | NM_011701.4 | ECM |
| Igf1 | NM_001111274.1 | M2 | Hprt | NM_013556.2 | Housekeeping |
| Il10 | NM_010548.2 | M2 | Tbp | NM_013684.3 | Housekeeping |
| Il12b | NM_001303244.1 | M1 | | | |

TABLE 2
Human nanoString gene panel

| Gene | Target Identifier | Annotation | Gene | Target Identifier | Annotation |
|---|---|---|---|---|---|
| Acta2 | NM_001613.1: 645 | ECM | Il6 | NM_000600.3: 364 | M1 |
| Aggf1 | NM_018046.3: 35 | Angiogenesis | Jag1 | NM_000214.2: 915 | Immune |
| | | | | | signaling |
| Ang | NM_001145.4: 949 | M2 | Lum | NM_002345.3: 1285 | ECM |
| Btg1 | NM_001731.2: 775 | Immune | Marco | NM_006770.3: 61 | M2 |
| | | signaling | | | |
| Ccl15 | NM_032965.4: 869 | Angiogenesis | Mmp2 | NM_004530.2: 2360 | M1 |
| Ccl18 | NM_002988.2: 585 | M2 | Mmap9 | NM_004994.2: 1530 | M2 |
| Ccl2 | NM_002982.3: 123 | M1 | Mrc1 | NM_002438.2: 525 | M2 |
| Ccl22 | NM_002990.3: 797 | M2 | Pdgfa | NM_002607.5: 2460 | Angiogenesis |
| Ccl24 | NM_002991.2: 18 | M2 | Pdgfb | NM_033016.2: 1480 | Angiogenesis |
| Ccl26 | NM_006072.4: 184 | M2 | Pdgfc | NM_016205.2: 2596 | Angiogenesis |
| Ccl5 | NM_002985.2: 280 | Angiogenesis | Pdgfra | NM_006206.3: 1925 | ECM |
| Ccl8 | NM_005623.2: 689 | M1 | Pdgfrb | NM_002609.3: 265 | ECM |
| Ccr7 | NM_001838.2: 1610 | M1 | Ramp1 | NM_005855.2: 200 | M2 |
| Cd163 | NM_004244.4: 1630 | M2 | Spp1 | NM_000582.2: 760 | Immune |
| | | | | | signaling |
| Cd80 | NM_005191.3: 674 | M1 | Stat3 | NM_003150.3: 2060 | Immune |
| | | | | | signaling |
| Clec10a | NM_182906.2: 430 | M2 | Stat6 | NM_003153.3: 2030 | Immune |
| | | | | | signaling |
| Col3a1 | NM_000090.3: 180 | ECM | Tgfb1 | NM_000660.3: 1260 | ECM |
| Ctnnb1 | NM_001098210.1: 1815 | Angiogenesis | Tie1 | NM_005424.2: 2610 | Angiogenesis |
| Cxcr4 | NM_003467.2: 1335 | Immune | Timp3 | NM_000362.4: 1640 | Immune |
| | | signaling | | | signaling |
| Egfl7 | NM_016215.3: 1252 | Angiogenesis | Tnf | NM_000594.2: 1010 | M1 |
| Ets1 | NM_005238.3: 1305 | Angiogenesis | Tnfrsf11a | NM_003839.3: 226 | M2 |
| Flt1 | NM_002019.4: 530 | Angiogenesis | Vcan | NM_004385.3: 9915 | ECM |
| Fn1 | NM_212482.1: 1776 | ECM | Vegfa | NM_001025366.1: 1325 | Angiogenesis |
| Foxo1 | NM_002015.3: 1526 | Angiogenesis | Vegfb | NM_003377.3: 687 | Angiogenesis |
| Foxo4 | NM_001170931.1: 1121 | Angiogenesis | Vegfc | NM_005429.2: 565 | Angiogenesis |
| Fyn | NM_002037.3: 765 | Immune | Vim | NM_003380.2: 694 | ECM |
| | | signaling | | | |
| Hspg1 | NM_005529.5: 2715 | ECM | Wnt5a | NM_003392.3: 475 | Immune |
| | | | | | signaling |
| Ido1 | NM_002164.5: 369 | M1 | Gapdh | NM_001256799.1: 386 | Housekeeping |
| Il1b | NM_000576.2: 840 | M1 | Tbp | NM_001172085.1: 587 | Housekeeping |

### Cell Viability

To examine potential cytotoxicity of drug and hydrogel components, RAW-Blue™ cells were plated at 5×10³ cells/well in 96 well plates. Media was replaced at 24 h containing either drug (100 μM to 0.03 μM in half-log dilutions) or polymeric components (Ad-HA or CDNP, 5 to 0.04% w/v in five-fold dilutions) of interest. After 24 h, metabolic activity was assessed by PrestoBlue™ (Fisher) following the manufacturer's protocol, n=3 per treatment group, Absorbance was recorded at 570 nm, background subtracted from cell-free control wells, and normalized to untreated controls.

### Surface Plasmon Resonance of Drug Binding to CD

Surface plasmon resonance (Nicoya, OpenSPR) was used to quantify binding affinity between CD and celastrol. The instrument was primed with running buffer (0.5% v/v DMSO in PBS), a high sensitivity carboxyl sensor was installed, and both channels were cleaned with 10 mM HCl at 150 μL/min. The surface of the sensor was activated via injection of 200 μL of 0.1 M EDC/NHS in DI water in both channels at 20 μL/min. The ligand, aminated β-cyclodextrin, prepared as previously described, was dissolved in sodium acetate buffer (10 mM, pH 6, 1.2 mg/mL) and immobilized on the surface in channel 2 at 20 μL/min. Residual succinyl esters were deactivated by ethanolamine. The analyte, celastrol, was dissolved in running buffer and injected at concentrations of 62.5 μM, 125 μM, and 250 μM. Between tests, the injection port was rinsed with 1 mL of running buffer. Curves were analyzed in GraphPad Prism 8 via the association kinetics model.

### Hydrogel Erosion and Drug Release

To determine the rate at which hydrogels were eroded, established methods of one-dimensional hydrogel degradation were used. Assays were performed in custom made acrylic erosion wells, having a hydrogel chamber (4.3 mm diameter, 7 mm depth) overlaid with a supernatant chamber (1.6 cm diameter, 10 mm depth). Hydrogels (n=4 per group, 30 μL each) were deposited in the hydrogel chamber, wells were centrifuged to provide an even hydrogel surface, and the hydrogel was covered with 1 mL of PBS. Samples were incubated at 37° C., and the supernatant was collected at regular intervals with replacement by fresh buffer. At the endpoint, hydrogels were degraded in 2 mg/mL hyaluronidase (Sigma) for complete sample recovery. Quantification of polymeric content in release buffer was performed via uronic acid assay. For each supernatant sample, 50 μL of the sample was combined with ice-cold sulfuric acid containing sodium tetraborate decahydrate (1 mL, 19 mg/mL). Samples were incubated at 100° C. for 10 mins and cooled on ice prior to addition of carbazole (30 μL, 1.25 mg/mL in ethanol). Samples were briefly vortexed, incubated at 100° C. (15 min), and cooled on ice prior to recording absorbance at 525 nm (Thermo, Spectronic BioMate 3). Data is presented as the cumulative erosion over time, normalized to total sample recovery after enzymatic degradation.

For release studies, hydrogels were prepared including celastrol (5 mM). Celastrol was loaded into CDNPs by overnight mixing in PBS prior to the addition of Ad-HA. Hydrogels (n=3 per group) were prepared and loaded into erosion wells as previously described, with 1 mL of RAW-Blue™ cell media used as the supernatant which was subsequently collected with replacement at set time points over 14 days. Samples were stored at −20° C. until analysis of bioactivity in RAW-Blue™ cells, which were plated at 25×10³ cells/well in 96 well plates. After 24 h, media was replaced with unconditioned media (control) or conditioned media from the release studies, supplemented with zymosan (100 μg/mL) as an inflammatory stimulus. Inflammatory activity was detected at 24 h after treatment using QUANTI-Blue™ Solution, following the manufacturer's protocol as described elsewhere herein.

### In Vitro Nanoparticle Uptake

For in vitro imaging of nanoparticle uptake, CDNPs were dissolved at 50 mg/mL in carbonate buffer (0.1 M, pH 8.5) and fluorescently labelled with Alexa Flour 555 NHS (10 μg/mL, Fisher). The reaction proceeded for 2 h at RT in the dark prior to product recovery by centrifugal filtration (10 kDa MWCO, Amicon). The product was repeatedly washed with water to remove unbound dye and lyophilized.

To determine if CDNPs released from hydrogels were uptaken by MF, hydrogels were prepared from the fluorescently labeled nanoparticle (CDNP-AF555) with or without celastrol inclusion (5 mM) and loaded into erosion wells as previously described (n=3 per group), with 1 mL of RAW-Blue™ cell media used as the supernatant which was collected after 48 hours. Conditioned media was transferred to RAW-Blue™ cells, seeded 24 hours prior at 15×10³ cells/well in a 96 well glass bottom plate. After 24 h, cells were washed by PBS, fixed with 4% paraformaldehyde (15 min, 37° C.), and stained for cell membrane (5 μg/mL AlexaFluor 488 wheat germ agglutinin, Fisher) and nuclei (NucBlue™, Fisher) for 15 min at room temperature. Plates were washed by PBS prior to imaging (Leica, DMI 6000B). CDNP-AF555 uptake was assessed in ImageJ, quantified as the integrated fluorescence density.

### Data Presentation and Statistical Analysis

Data presented are mean±standard deviation (SD), unless otherwise indicated.

Statistical analysis was performed using GraphPad Prism v9.3.1 and determined by analysis of variance (ANOVA), using repeated measurements where appropriate in conjunction with post-hoc Tukey's honest significant difference test. Normality was assessed by Shapiro-Wilk test. For two-way ANOVA, the Geisser-Greenhouse's epsilon correction was applied. Significance was determined at p<0.05.

### Example 1: Development and Characterization of Cyclodextrin Nanoparticles (CDNPs)

Cyclodextrins are versatile macrocyclic hosts, used in the pharmaceutical industry as excipients to improve drug solubility and bioavailability, a use which has been meaningfully extended through the development of nano- and bulk-materials that leverage this host capacity for drug retention, including for macrophage (MF)-targeted therapies. In biomaterial applications, the transient guest-host complexes formed between CD and hydrophobic guest molecules is also a common means of forming dynamically crosslinked hydrogels. For this purpose, adamantane (Ad) is frequently used, as its size readily complements that of CD's hydrophobic cavity, contributing to formation of a one-to-one inclusion complex with relatively high affinity (Keq=10⁵ M). Here, CD's versatile host capacity is leveraged to develop a nanotherapeutic drug delivery vehicle which is dynamically crosslinked by a host-modified polymer.

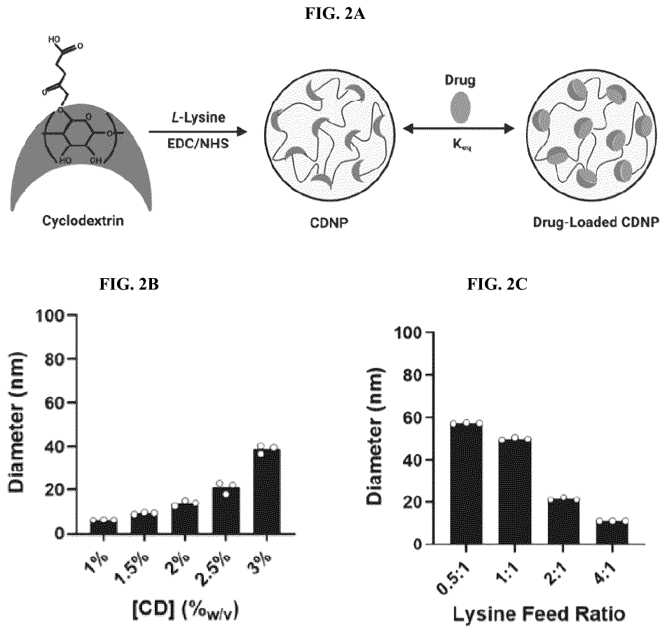

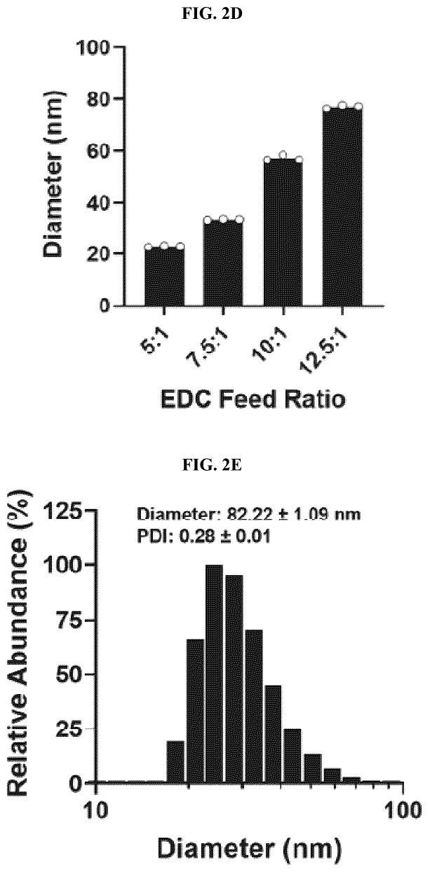

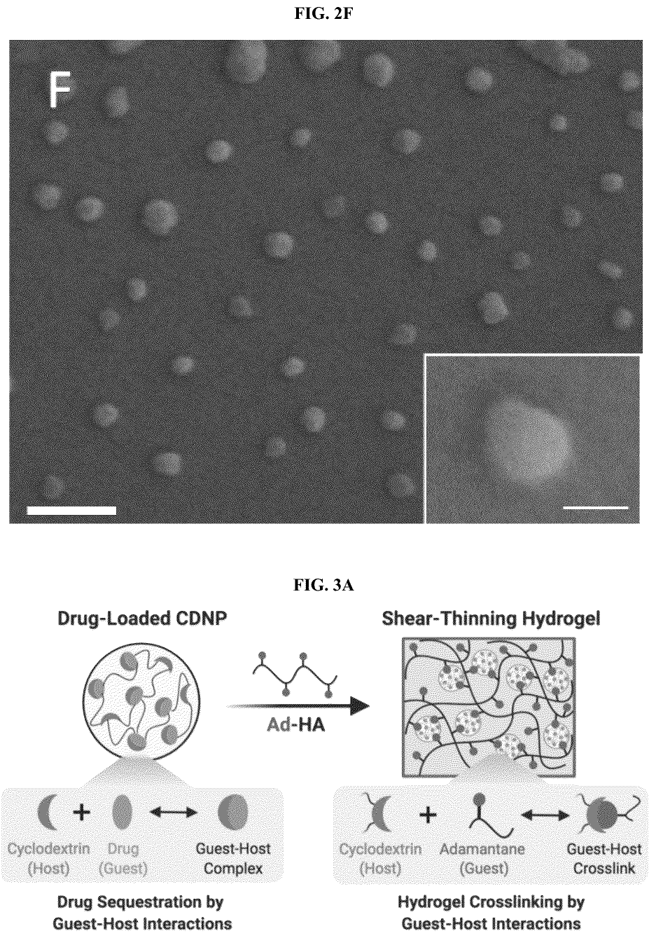

To prepare cyclodextrin nanoparticles (CDNPs) of varying size, particles were formed through EDC/NHS mediated amidation between succinyl-β-cyclodextrin and L-lysine (FIG. 2A). While nanoparticles previously formed by these methods were approximately 30 nm in diameter, it was rationalized that an increased particle size would afford both a greater surface area for polymer-NP crosslinking as well as an improved volume to surface area ratio essential to forming a discrete drug reservoir. Synthesis conditions were therefore explored as a means to control CDNP size, where related variables include the concentration of CD during crosslinking (FIG. 2B), the ratio of lysine to succinyl groups (i.e., lysine feed ratio, FIG. 2C), and the catalyst concentration relative to succinyl groups (i.e., EDC feed ratio, FIG. 2D). Nanoparticle size was positively correlated with the substrate and catalyst concentrations. Furthermore, a 1:1 ratio of amine to succinate at a lysine feed ratio of 0.5:1 resulted in the largest observed diameter.

Further increase in the particle size was not observed, as increased substrate or catalyst concentrations resulted in formation of a solid hydrogel during the reaction. Thus, the largest available CDNPs were pursued for hydrogel development; nanoparticles formed with 3.3% w/v CD, a 0.5:1 lysine feed ratio, and a 12.5:1 EDC feed ratio reliably produced CDNPs with a diameter of 80 nm, as confirmed by dynamic light scattering (DLS, FIG. 2E) and scanning electron microscopy (SEM, FIG. 2F). Notably, nanoparticles on the order of 50-100 nm are likewise preferential for phagocytic uptake necessary for subsequent MF-targeted delivery applications.

### Example 2: Polymer-Nanoparticle Interactions Form a Shear-Thinning and Self-Healing Hydrogel

Development a shear-thinning and self-healing hydrogel was sought by leveraging the high affinity supramolecular interaction of CD at the nanoparticle surface with Ad. Here, hyaluronic acid (HA, MW=82 kDa or 337 kDa) was exchanged against Dowex-100 resin and neutralized by tetrabutylammonium hydroxide to yield HA-TBA (FIGS. 9A-9B), required for subsequent anhydrous reaction. HA was pendantly modified by 1-adamantane acetic acid, where the degree of substitution was controlled by altering the molar ratio of Boc₂O catalyst to HA repeat units. The guest-modified polymers were prepared having a range of functionalization determined by ¹H-NMR (FIGS. 10A-10D): 10% (337 kDa Ad-HA (Low)), 18% (82 kDa Ad-HA (Low)), and 43% (82 kDa Ad-HA (High)). These degrees of substitution were selected to allow for variation in guest-host crosslink density between 82 kDa low and high modifications. Additionally, varying polymer molecular weight affords a similar number of potential crosslinking sites between high and low molecular weight polymers, where 337 kDa Ad-HA (Low) and 82 kDa Ad-HA (High) have an average of 85 and 88 Ad per HA macromer, respectively. The higher molecular weight polymer, however, may better span inter-particle distance to facilitate subsequent hydrogel formation. The relative importance of guest-modification density and polymer molecular weight were subsequently investigated.

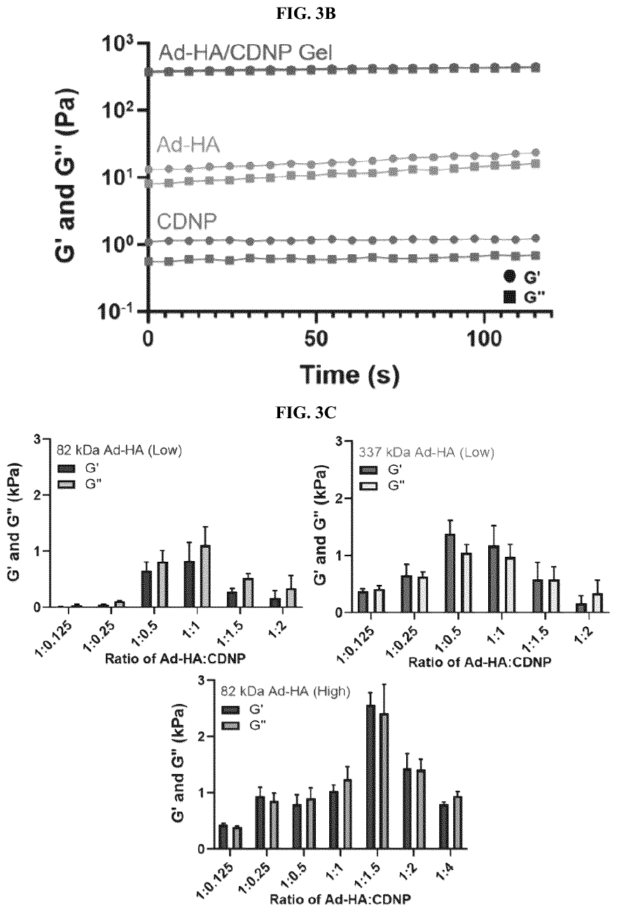

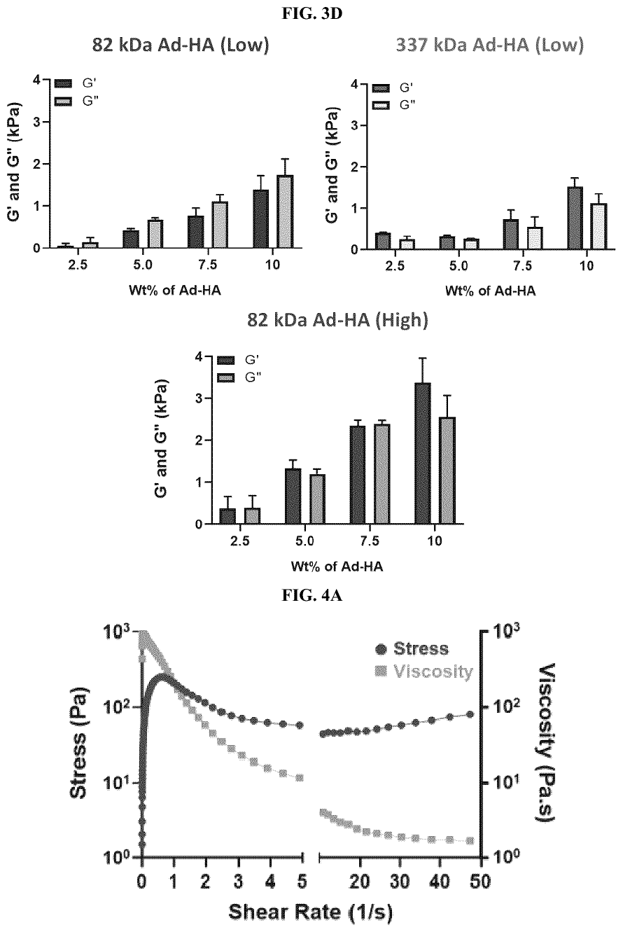

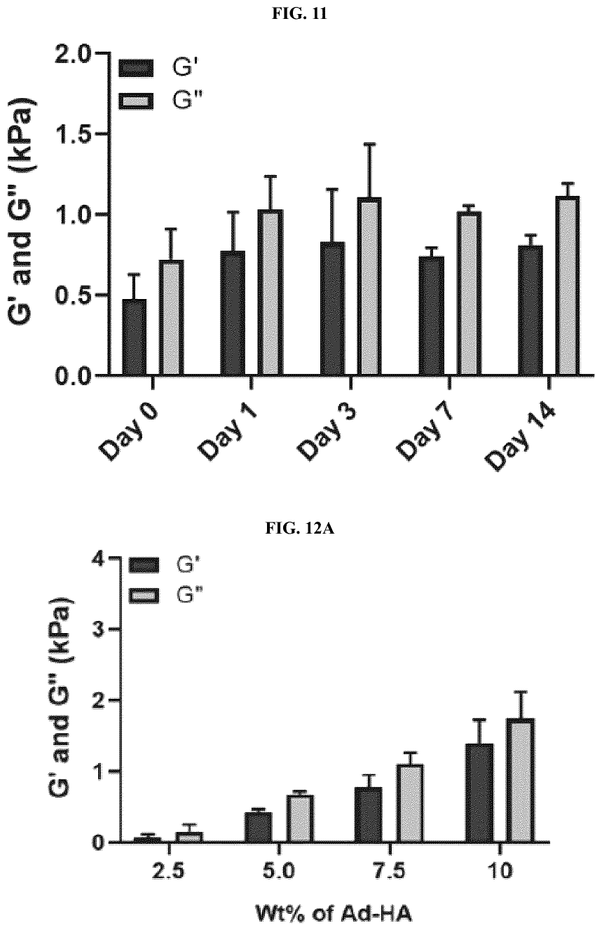

The guest-polymers prepared serve as a crosslinker between host (CDNP) particles to yield polymer-nanoparticle hydrogels that self-assemble through guest-host crosslink formation (FIG. 3A). Separate components, Ad-HA and CDNP, were initially viscous solutions. After their mixing, rapid formation of a viscoelastic solid was qualitatively observed, as was an increase in the storage (G′) and loss (G″) moduli by several orders of magnitude (FIG. 3B). Interestingly, the hydrogel properties were time dependent, exhibiting increases in moduli after room temperature incubation over several days. Without wishing to be bound by any theory, it has been reasoned that, while individual guest-host complex formation is rapid, it is likely that polymer relaxation is required for thermodynamic equilibrium to be achieved. To quantitatively investigate this annealing behavior, rheological properties were assessed over a span of 14 days (FIG. 11). A moderate increase in moduli was observed up to day 3, with subsequent stabilization thereafter. For following studies, hydrogels were therefore annealed for 3 days prior to evaluation.

The viscoelastic properties of the hydrogels was further investigated via oscillatory shear rheology. As the number of host sites accessible for guest-polymer binding at the nanoparticle surface is indeterminate, the ratio of guest to host components was empirically investigated (FIG. 3C). At high ratios of Ad-HA:CDNP, low moduli were observed, potentially due to polymeric coating of the CDNPs as opposed to desirable interparticle crosslinking. Conversely, high ratios of Ad-HA:CDNP likewise formed hydrogels with low moduli, presumably due to a low density of guest-host interactions required for stable gel formation. Greatest moduli were observed for an intermediate polymer to nanoparticle ratio, which were dependent on polymer modification and molecular weight.

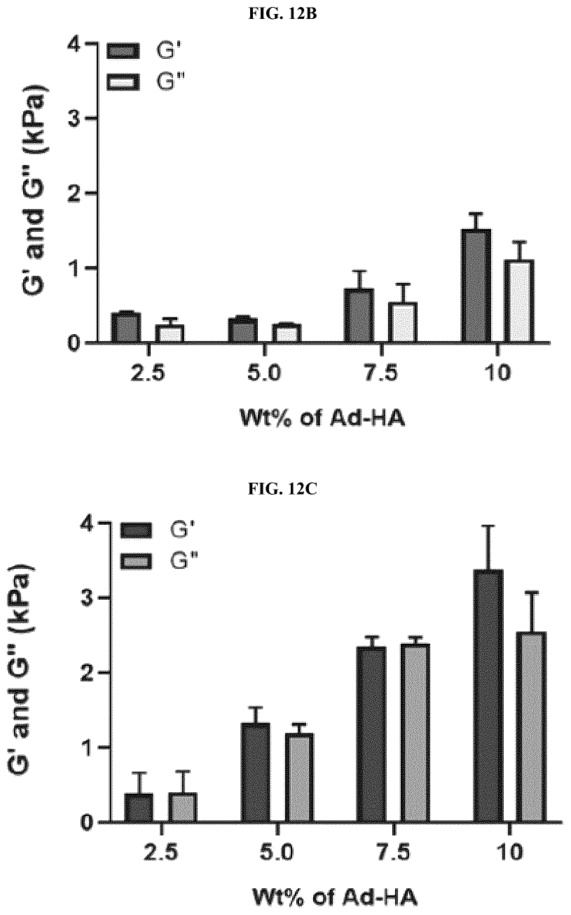

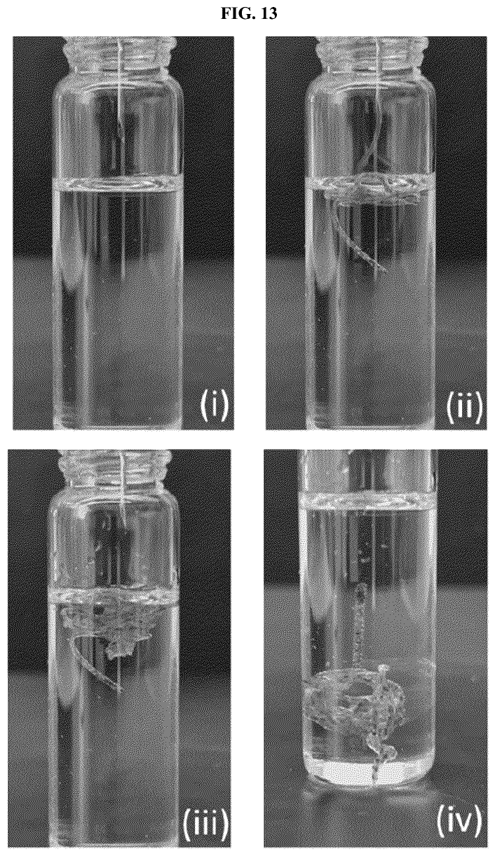

Across these datasets, it is apparent that crosslink density is critical to stable gel formation as increased polymer modification yielded increased hydrogel moduli when molecular weight remained constant. Consistent with these observations, hydrogel moduli were likewise dependent on hydrogel concentration (FIGS. 12A-12C). Moreover, increasing polymer molecular weight afforded hydrogels with moderately increased moduli, relative to 82 kDa Ad-HA (Low). However, most robust hydrogel formation was observed at a relatively high ratio of Ad-HA:CDNP which is undesirable for later drug-loading applications. Hydrogels composed of 82 kDa Ad-HA (High) exhibited the most robust moduli (G′=2.72±0.018 kPa) at a 1:1.5 ratio of Ad-HA:CDNP and provide a relatively high CDNP content essential for later therapeutic delivery.

In sum, these studies demonstrate a method of tuning the mechanical properties of associative polymer-nanoparticle hydrogels and highlight the dominant role of dynamic bond density as a critical factor in stable hydrogel formation. More specifically, these studies revealed that 82 kDa Ad-HA (High) affords the most robust hydrogel formation and does so at polymer-nanoparticle ratios that are beneficial for subsequent drug loading.

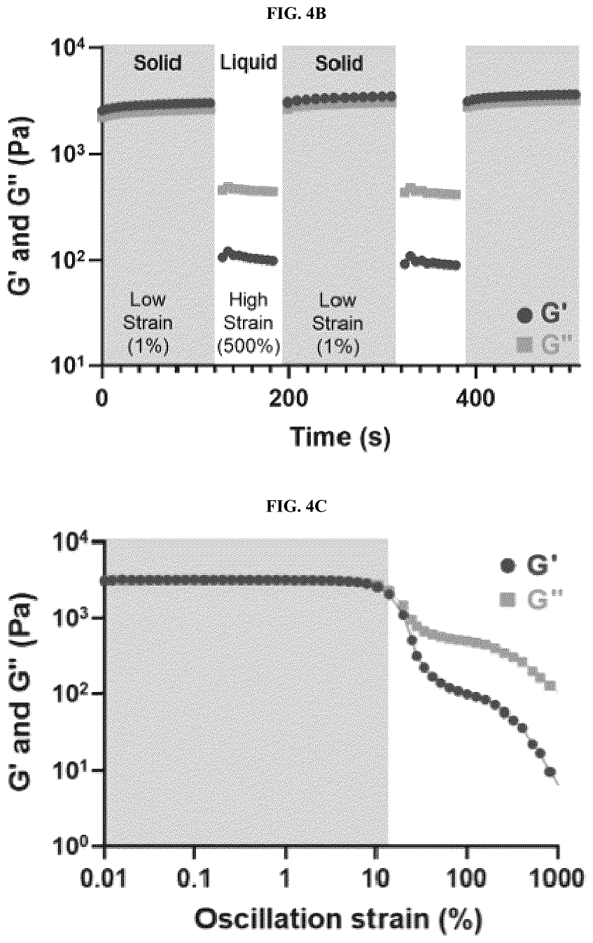

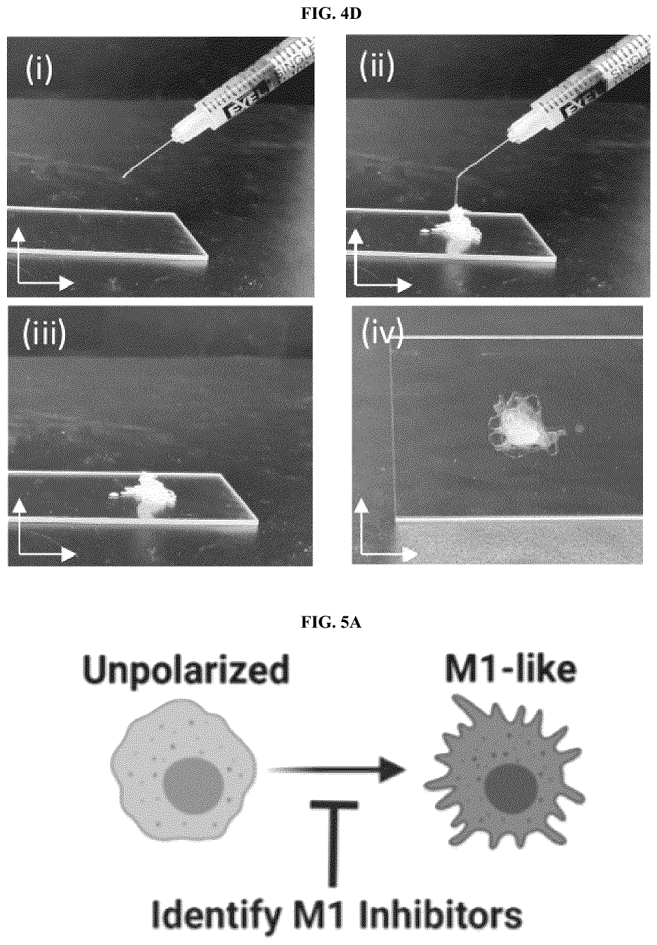

Hydrogels formed through dynamic bonds assume varying structural integrity under strained conditions. As the bonds are continually broken by the application of sufficient external force, bond disassembly enables fluid-like flow and shear-thinning hydrogel injection. Continuous flow experiments (FIG. 4A) were used to examine viscosity and stress over an increasing shear rate. As expected for shear-thinning materials, the hydrogels displayed reduced viscosity at high shear-rates and a resulting plateau in the observed shear stress. To examine the potential for rapid self-healing as result of dynamic bond self-healing, hydrogels were subject to repeated cycles of high (500%) and low (1%) strain, representing processes of disassembly when injected through a syringe needle (shear-thinning) and subsequent re-assembling (self-healing). Under high strain conditions, fluid-like behavior (G″>G′) was observed, with recovery of solid properties observed within seconds after onset of low strain conditions (FIG. 4B). Results are consistent with oscillatory strain sweeps (0.01%-1000% strain; 10 Hz) wherein a yield stress of approximately 10% was observed, above which the hydrogels underwent forceful disassembly and fluid-like behavior (FIG. 4C).

In sum, the hydrogels exhibit fluid-like flow under high strain for ease of injection (shear-thinning). When the strain is removed, the hydrogels rapidly recover such as to allow for retention within the tissue. As a result, hydrogels could be pre-formed within a syringe and easily injected as liquids that rapidly re-solidify (FIG. 4D and FIG. 13).

### Example 3: Drug Screening to Identify Immunomodulatory Small Molecule Drugs

Identification of small molecule drugs capable of modulating MF phenotype were next sought for application in the context of tissue injury and inflammatory disease. Specifically, identification of a candidate that could suppress the damaging M1-like MF phenotype following injury and promote the reparatory M2-like MF phenotype for injury resolution was pursued. Therefore, a two-step screening process was developed that first identified pharmacological inhibitors of M1-like transcription using RAW-Blue™ cells, which are a readily available and inexpensive reporter cell line that incorporates a SEAP reporter construct downstream of both AP-1 and NF-κB promoter regions, and thus reports pro-inflammatory activation. The cell line is frequently used to evaluate both agonists and antagonists of M1-like polarization. While some approaches have been successful in identifying compounds for MF re-polarization such as through morphological analysis, applications have focused on redirection towards an M1 and not an M2 state. Currently, few approaches exist to directly assay for M2-like polarization. In the second step of the screening process, transcriptional analysis, via qPCR and multiplex analysis (nanoString), was used to identify compounds that promote canonical M2-like MF gene expression.

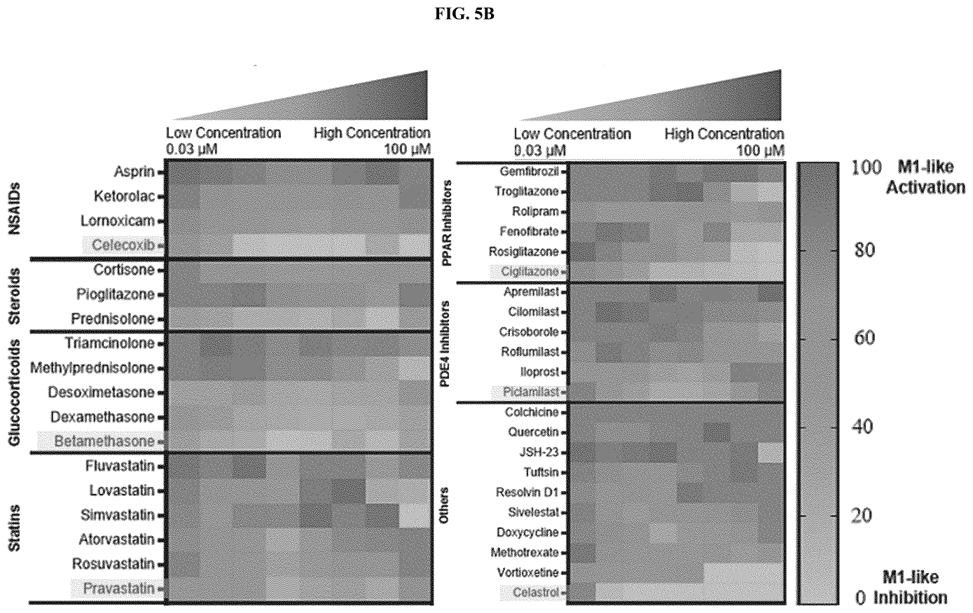

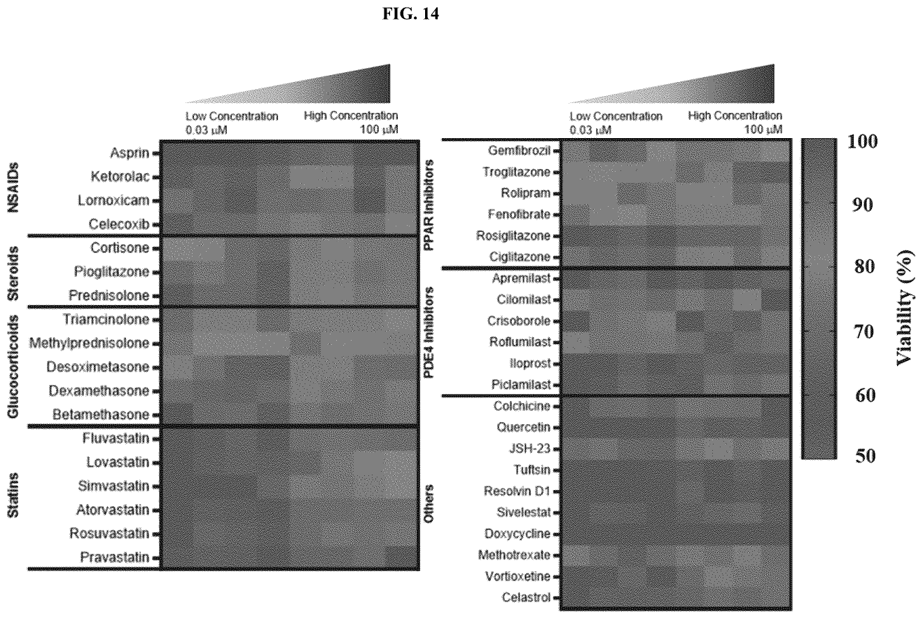

Initial drug screens were approached through an unbiased evaluation that compared dozens of drugs across multiple drug classes as such direct comparison is currently lacking from literature. Suppression of M1-like activation was therefore examined for a targeted library of 45 small molecule drug candidates that span a diverse set of specific drugs and drug classes that have established immunosuppressive or immunomodulatory capacity, including glucocorticoids, statins, and PPAR inhibitors among others. For primary evaluation in RAW-Blue™ cells, cells were stimulated by zymosan, a toll-like receptor 2 (TLR2) agonist that mimics sterile inflammation, and concurrently treated with the drugs of interest. HDAC inhibitors examined had no effect and were excluded from the data presented and subsequent studies. Other drug classes had varying degrees of efficacy (FIGS. 5A-5B), with select drugs exhibiting sub-micromolar inhibition of M1-like activation. Viability of RAW264.7 cells was further examined following drug treatments (FIG. 14). Notable losses of viability were not observed; initial results are therefore a direct result of desired pathway inhibition and not an artifact of drug toxicity. Within each class, drugs were rank-ordered based on the largest cumulative suppression of M1-like activation across doses, and the most potent drug from each class was selected for further in vitro phenotyping studies.

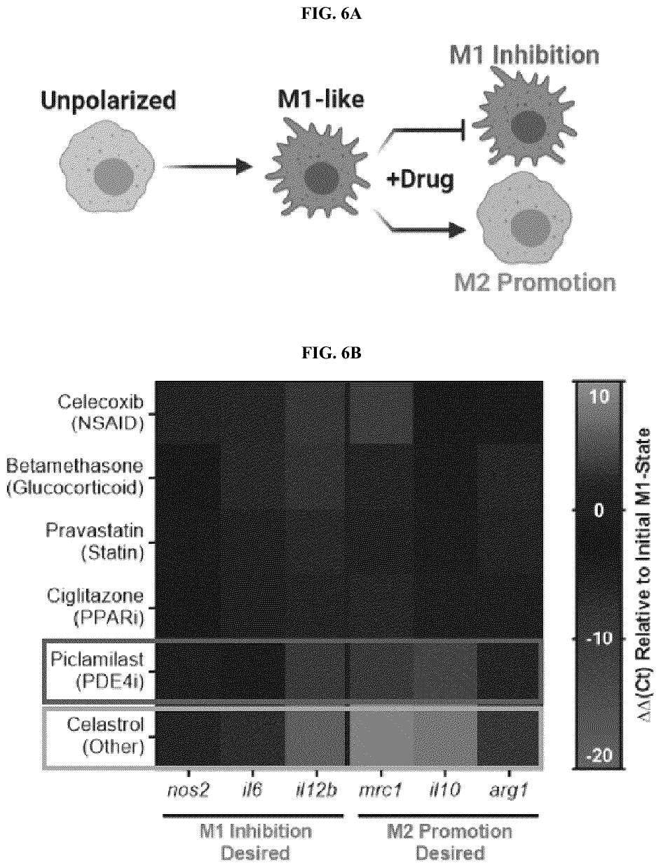

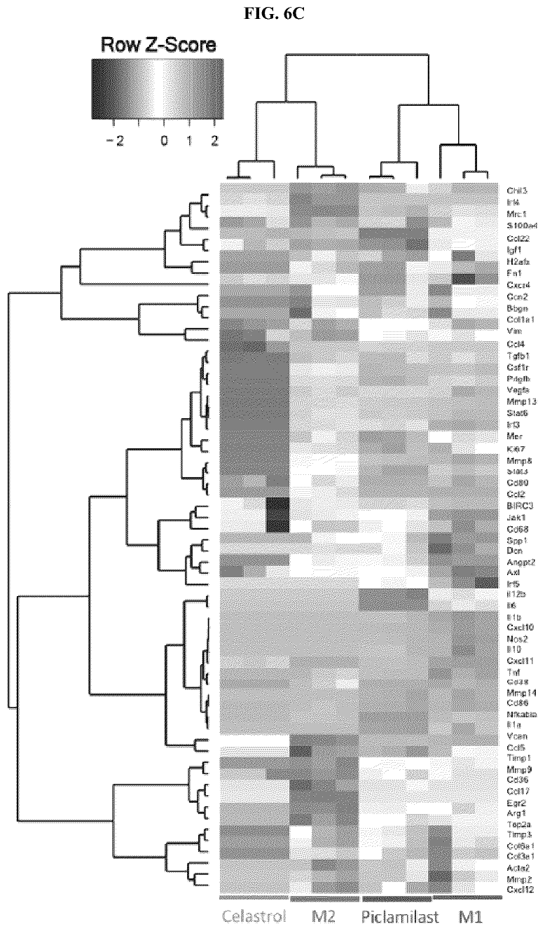

After initial inhibitory screening, the effects of six selected drugs (i.e., celecoxib, betamethasone, pravastatin, ciglitazone, piclamilast, and celastrol) on MF phenotype were further scrutinized by transcriptional analysis in zymosan-activated RAW264.7 MF in order to determine if these drug candidates further promote M2-activation. Canonical markers of both M1-like (nos2, i16, il12b) and M2-like (mrc1, arg1, il10) phenotypes were included (FIGS. 6A-6B). Two drugs, piclamilast and celastrol, significantly downregulated M1-like genes with concurrent upregulation of M2-like genes. Notably, these drugs showed the greatest decrease in il12b, a pro-inflammatory cytokine critical to adaptive immune activation, as well as the greatest increase in mrc1 and il10. MRC1 (CD206) is an established marker of M2-like activation, both in humans and mice that is correlated with decreases in MF migration and serum levels of inflammatory proteins, whereas IL10 is a potent anti-inflammatory cytokine critical towards wound healing and tissue repair. The relatively simple two-step screening approach therefore identified two drug candidates that exhibit potent anti-inflammatory action via high-throughput reporter screens, and a subset of the best-in-class drug candidates (piclamilast and celastrol) biased cells towards an M2-like activation state even during opposing pressure by TLR agonization.

Further in-depth transcriptional analysis of the two drugs candidates was performed by multiplex gene expression analysis (nanoString) in zymosan-activated bone marrow derived MF (BMDMs) treated with piclamilast and celastrol. The analysis measured gene expression across 91-genes that represent multiple M1-like and M2-like markers, as well as genes associated with ECM regulation, fibroblast activity, angiogenesis, and immune signaling pathways (Table 1).

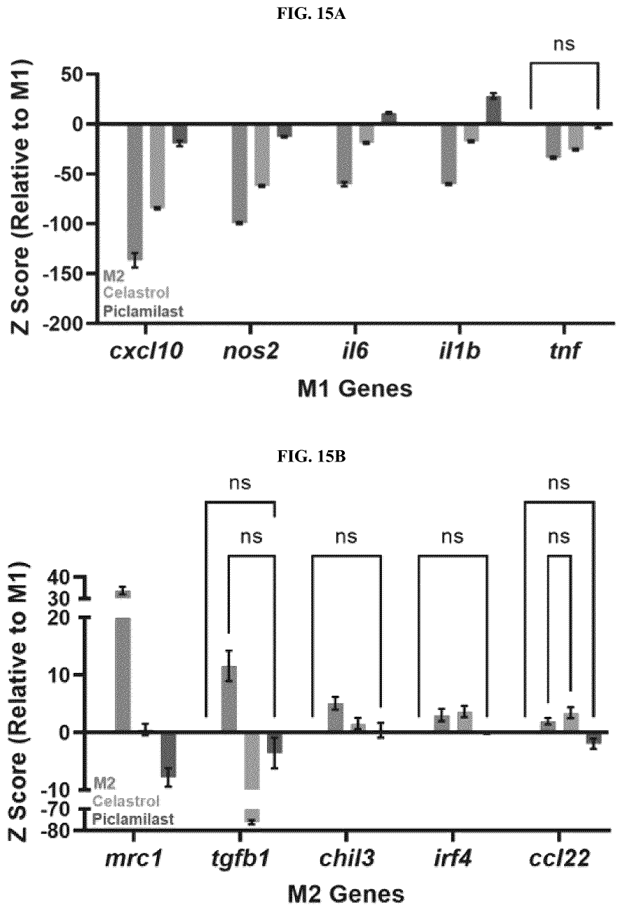

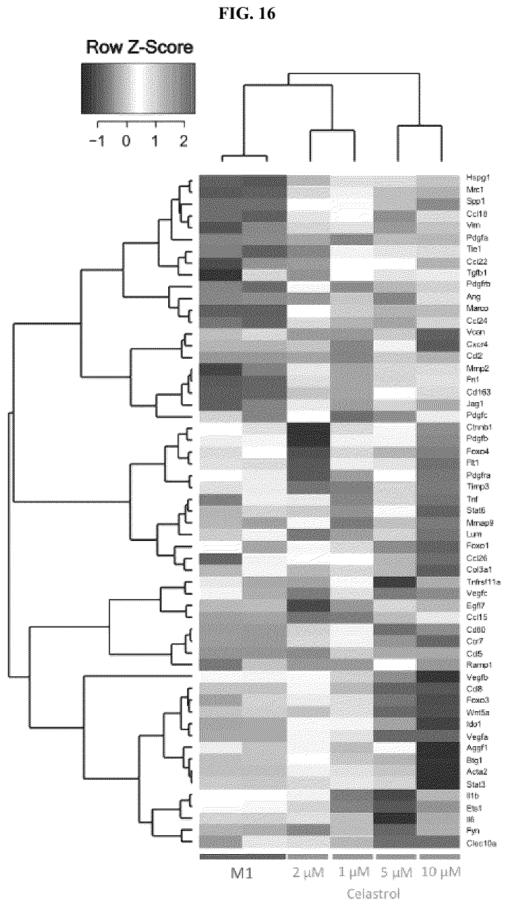

Cluster analysis and corresponding dendograms (FIG. 6C) most closely associate celastrol treatment with M2 controls, while piclamilast treatment was intermediate to M1 and M2 control phenotypes. Relative to M1 controls, downregulation of M1-associated genes was typically greater for celastrol than for piclamilast, with expression levels approaching or in some cases exceeding that of M2-like (IL-4 treated) controls (FIG. 15A). A number of these genes are implicated in chronic inflammation and impaired tissue healing. For example, CXCL10 (IP-10) is a MF-derived chemokine critical for the recruitment of inflammatory T-cell subsets. Celastrol likewise downregulated pro-inflammatory cytokines, including tnf, il1b, and il6 that perpetuate continued tissue injury and are common biomarkers of organ failure. Celastrol also preferentially modulated the expression of M2-associated genes (FIG. 15B). For example, celastrol upregulated irf4 and cc122 to levels consistent with M2-like controls; IRF-4 suppresses innate immune signaling through inhibition of the TLR/MyD88 pathway. Interestingly, celastrol treatment also drastically downregulated tgfb1, consistent with prior indications of its use as an antifibrotic agent and further motivating a potential use in tissue injury applications. For these reasons, celastrol was chosen as the final drug of interest for continuing studies.

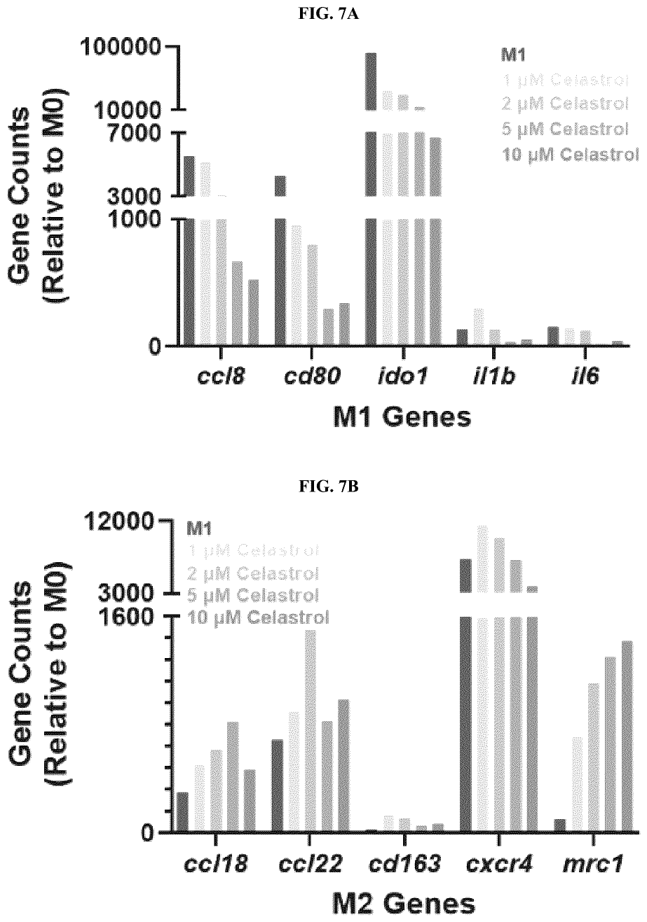

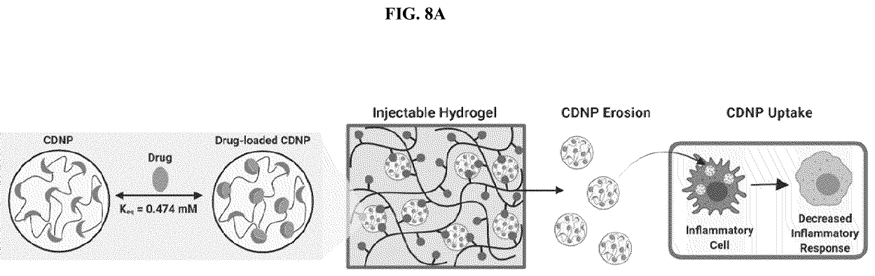

While results in cell lines and primary murine cells are promising, there exist nuanced differences between the murine and human inflammatory programs that may hinder translation and warrant early investigation. To ensure drug activity across species, transcriptional analysis was further performed in LPS-activated MF derived from peripheral blood mononuclear cells (PBMCs) using a panel of human genes (Table 2). The choice of LPS instead of zymosan as a pro-inflammatory stimulus was made to allow comparison to other studies of human MF, which more commonly use LPS. Subsequent to LPS activation, cells were treated with varying concentrations of celastrol (1-10 μM). Both cluster analysis (FIG. 16) and expression of specific genes (FIGS. 7A-7B) indicated dose response across celastrol concentrations, with desired upregulation of M2-associated genes and downregulation of M1-associated genes when compared to LPS-treated controls. These include, for example, decreased expression of the T-cell costimulatory signal cd80 that is critical for TH1 activation. The expression of inflammatory chemokines (ccl8) and cytokines (il1b, i16), and immunoregulatory enzymes (idol) were likewise suppressed in a dose-dependent fashion. In contrast, the expression of M2-associated genes was typically upregulated, including for cell surface markers (mrc1) as well as chemokines and their receptors (ccl18, cc122, cxcr4). Overall, these findings are consistent with the observations in murine cell lines and primary cells, indicating the immunomodulatory capacity of celastrol is not species specific.

### Example 4: Drug Encapsulation and Release

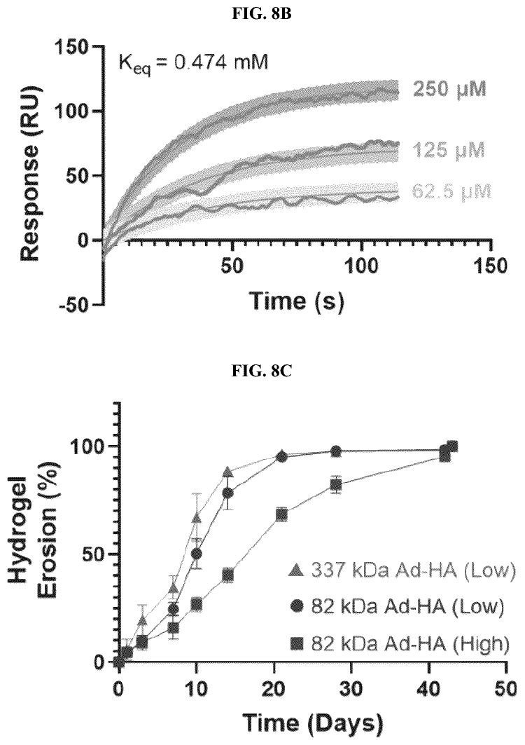

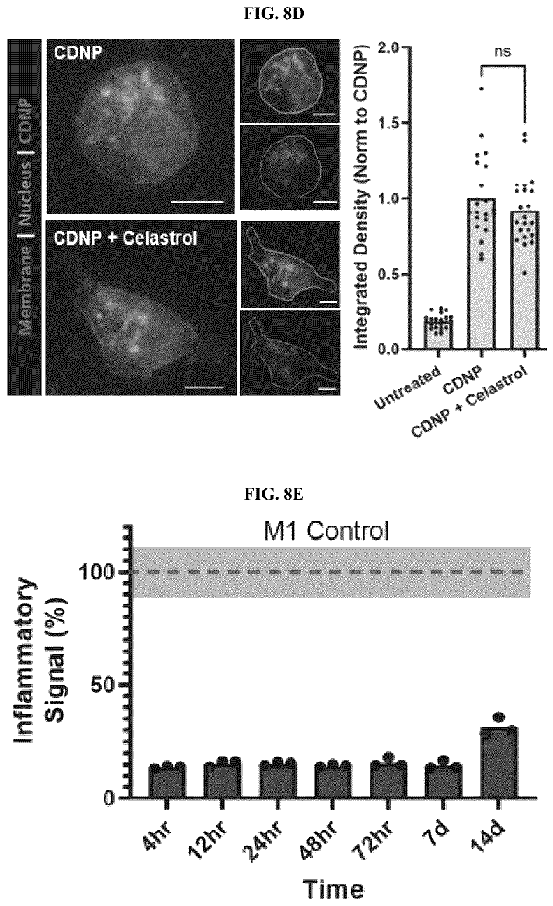

While celastrol has moderate oral bioavailability which can be enhanced by formulation, it displays significant sex-dependences in systemic bioavailability and such administration is associated with hepatotoxicity, hematopoietic system toxicity, nephrotoxicity, and undesirable biodistribution. Moreover, local biomaterial-delivery strategies afford an opportunity to reduce clearance rates, prolong release, concentrate drug dose at the site of action, and enhance cell-specific delivery. For these reasons, examination of its potential as a suitable cargo for CDNP encapsulation and delivery from the polymer-nanoparticle hydrogels is described herein (FIG. 8A). CD is amenable to guest-host complex formation with a variety of hydrophobic small molecules, particularly polycyclic and aromatic structures with suitable size for host inclusion. In guest-host delivery systems, the diffusive release of guest drug molecules is prolonged by complex dissociation and subsequent re-association events that can sustain release for a period of weeks to months. The initial objective was to confirm whether celastrol is a suitable molecular guest for complexation with CD, as necessary for inclusion within the CDNP drug carrier. The equilibrium binding constant (Keq) was determined to be 0.474 mM by equilibrium analysis using surface plasmon resonance (FIG. 8B). These results are consistent with other polyaromatic guests, including tryptophan and doxorubicin, which are typically reported to have single micromolar Keq that is sufficient for drug delivery applications.

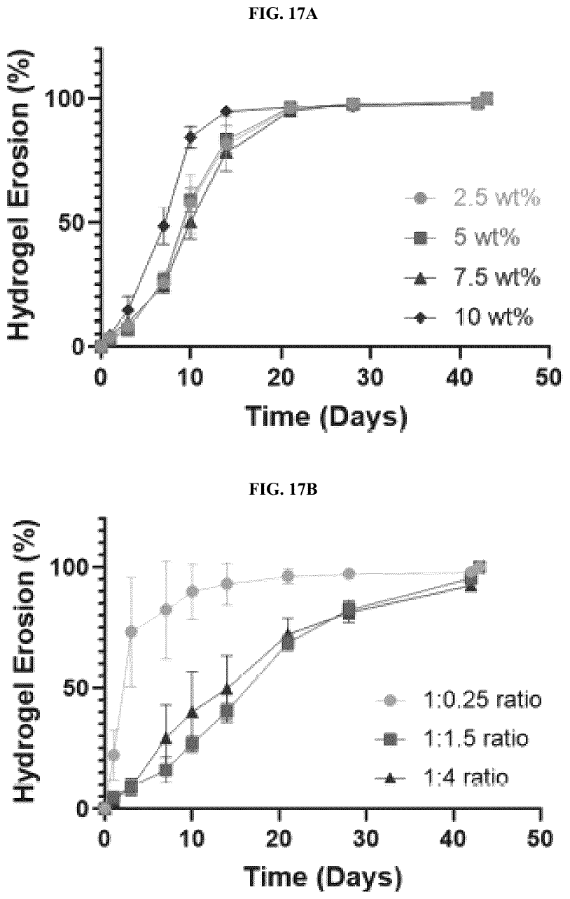

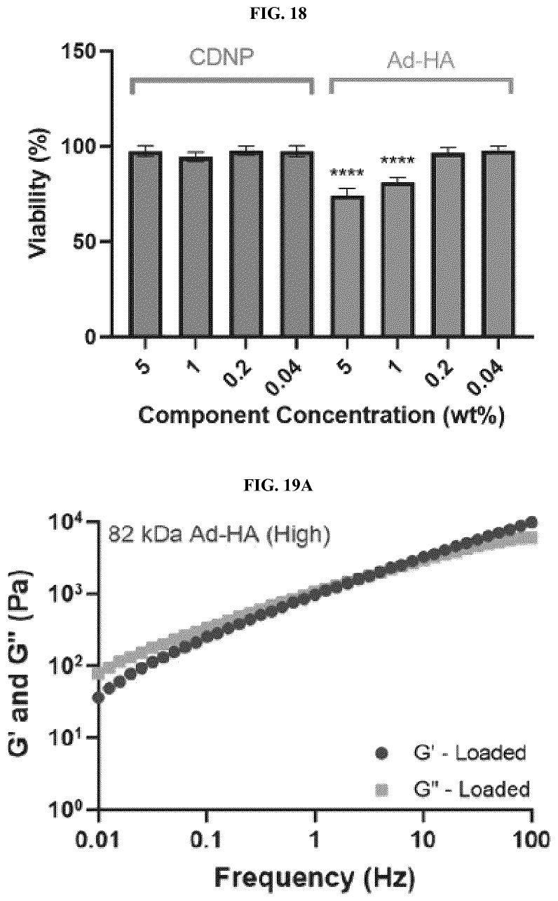

Supramolecularly assembled hydrogels typically degrade via surface erosion, with dependence on network structure and composition. It was therefore anticipated that nanoparticle release from the hydrogels would be both a means of hydrogel degradation and a potential method of cell-targeted delivery, owing to the rapid uptake of CDNPs by phagocytic immune cells that occurs both in vitro and in vivo. Hydrogel erosion was assessed over a six week period at 37° C., spanning a range of hydrogel compositions. Across these formulations, hydrogels typically exhibited slow erosion over the course of greater than one month, with 82 kDa Ad-HA (High) degrading slower than less effectively crosslinked network architectures (FIG. 8C). Degradation profiles were relatively independent of polymer concentration, but more highly dependent on ratio of guest to host components (FIGS. 17A-17B). Notably, reduced relative CDNP concentrations led to rapid erosion, attributable to insufficient hydrogel crosslinking. The release of soluble polymer by either degradation or erosion mechanisms warrants investigation of polymer cytocompatibility. Cells were therefore treated with media containing the separate hydrogel components, Ad-HA and CDNP (FIG. 18). Cell viability was unaffected by soluble CDNP. While relatively high concentrations of soluble Ad-HA (>1.0% w/v) significantly reduced cell viability, effects were moderate and observed only at concentrations in excess of what would result from hydrogel erosion. These results are in agreement with the literature concerning the use of guest-host hydrogels composed of Ad-HA for both in vitro and in vivo studies without detriment to exposed cells or tissues.

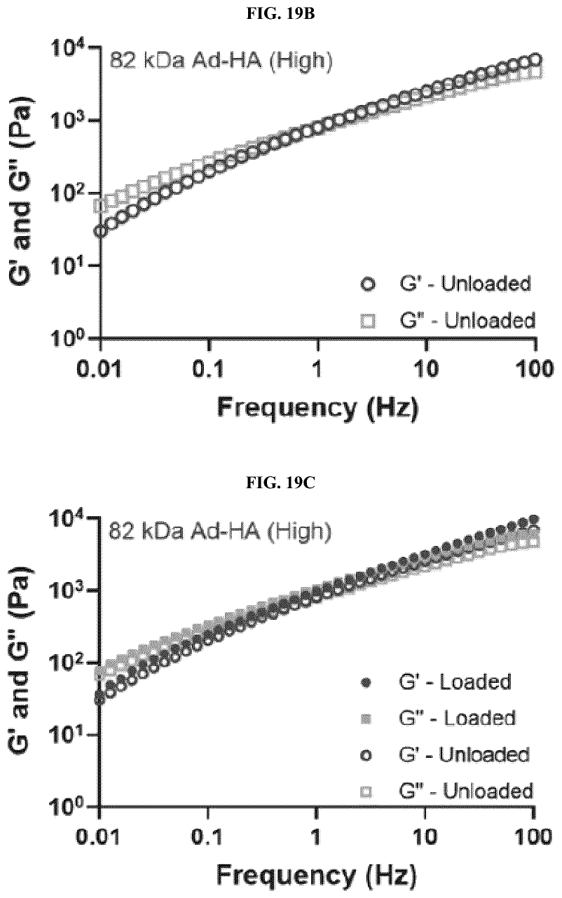

Celastrol-loaded hydrogel formulations were next prepared by simple mixing of celastrol (Cel) and CDNP under aqueous conditions and the resulting drug-loaded nanoparticle (CDNP-Cel) were used directly in hydrogel formation. Importantly, drug incorporation did not alter the rheological properties (FIGS. 19A-19C), including across all hydrogel formulations examined. Reductions in moduli might otherwise indicate disruption of the guest-host mediated crosslinking mechanism, such as by competitive binding. The absence of perturbation in the crosslinking mechanism is attributable to the disparity in binding affinity between Ad and celastrol.

Next, the ability of CDNP-Cel to be released from the hydrogels by erosion and subsequently uptaken my MF was assessed. For this purpose, CDNPs were fluorescently labeled by Alexa Fluor555 (CDNP-AF555). Both drug loaded and unloaded CDNP-AF555 nanoparticles were formulated in 82 kDa Ad-HA (High) hydrogels that were subsequently allowed to erode in media. RAW264.7 cells were treated with the conditioned media prior to imaging and quantification (FIG. 18D). Cells exhibited punctate accumulation of CDNP-AF555, consistent with prior observations of rapid CDNP uptake and endolysosomal accumulation in phagocytic immune cells. While MF polarization has been noted to impact nanoparticle uptake, CDNP-AF555 uptake was unhindered by celastrol loading. The release of CDNP-Cel by hydrogel erosion is therefore an efficient route of cell-targeted drug delivery from the injectable scaffold.

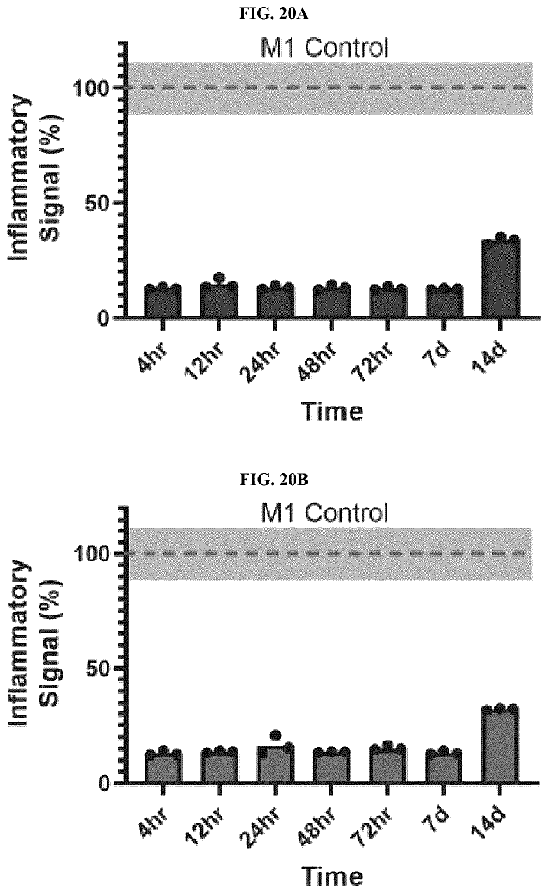

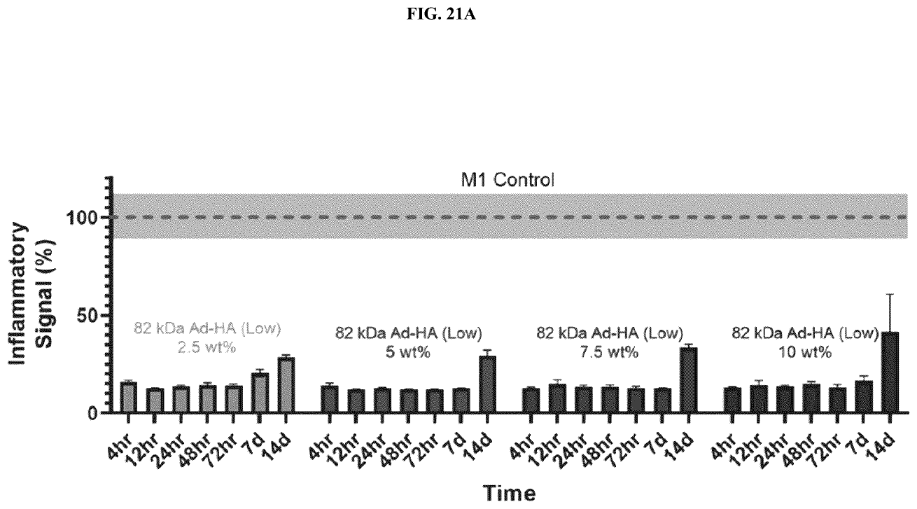

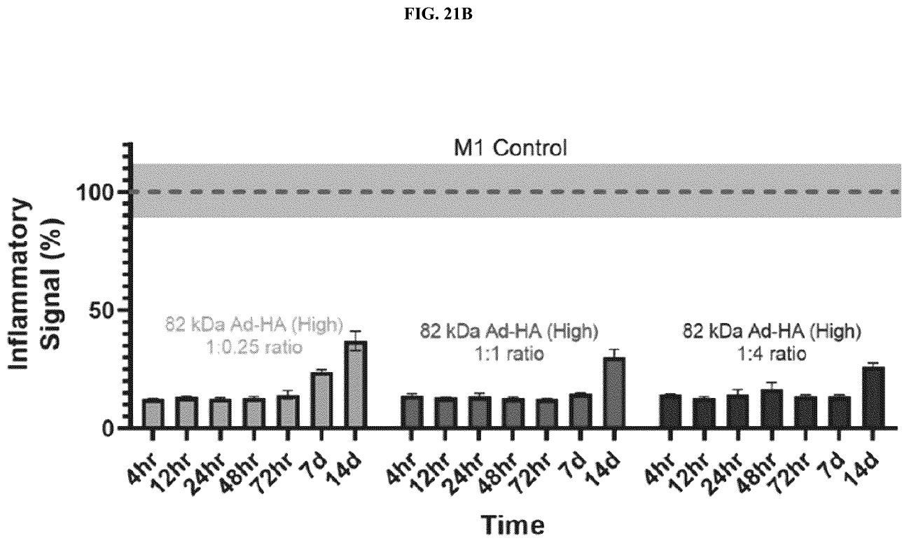

The final objective was to investigate the ability of the drug-laden hydrogels to provide bioactive concentrations of CDNP-Cel release, capable of modulating cell phenotype over time in vitro (FIG. 18E). Media was again conditioned by the degradation of celastrol-loaded hydrogels over a period of two weeks. RAW-Blue™ cells were concurrently stimulated with zymosan and treated with the conditioned media. Inflammatory pathway transduction was attenuated by >80% for one week and more moderately out to day 14. Similar results were observed for drug release from 82 kDa Ad-HA (Low) and 337 kDa Ad-HA (Low) hydrogels (FIGS. 20A-20B). Both diffusive and erosion processes likely contribute to celastrol release, as has been observed in other polymer-nanoparticle composites with encapsulated protein cargo. For hydrogel formulations with a reduced crosslinking density, such as those formed at reduced polymer or CDNP concentration, inhibitory drug affects waned earlier with moderate recovery of inflammatory signaling beginning at days 3-7 (FIGS. 21A-21B). This attributed to exhaustion of CDNP-Cel supply within the hydrogel depot, consistent with the observed rapid erosion of these formulations, as well as an inability to retain the drug within the hydrogel in the case of reduced CDNP concentrations.

Overall, these findings demonstrate the release of CDNP-Cel from the hydrogels is a promising strategy for MF-targeted drug delivery that can bias cell phenotype over the course of weeks. This timespan coincides well with the established timeline of exuberant inflammation that frequently underlies failed tissue repair programs, particularly in organ injury, and is therefore a promising immunoregenerative strategy towards such applications.

### Enumerated Embodiments

The following exemplary embodiments are provided, the numbering of which is not to be construed as designating levels of importance:

Embodiment 1 provides a hydrogel composition comprising:
(a) a nanoparticle comprising a plurality of macrocycles, wherein each of the macrocycles is independently covalently linked, either directly or indirectly, to at least one nanoparticle linker;
(b) a functionalized polymer comprising a hydrophilic polymer core substituted with a plurality of independently selected hydrophobic substituents, wherein at least a portion of the hydrophobic substituents are independently non-covalently associated with one of the macrocycles of the nanoparticle; and
(c) at least one therapeutic agent, wherein the at least one therapeutic agent is non-covalently associated with a macrocycle of the nanoparticle.

Embodiment 2 provides the hydrogel composition of Embodiment 1, wherein the least one nanoparticle linker independently comprises a moiety of Formula (I):

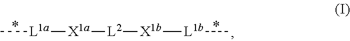

wherein:
L^{1a} and L^{1b} each independently comprise at least one moiety selected from the group consisting of —C(═O)—, -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
L² comprises at least one moiety selected from the group consisting of -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
X^{1a} and X^{1b} are each independently selected from the group consisting of —N(R′)—, —O—, and —S—;
each occurrence of R¹ is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl;
each bond [symbol] comprises a bond between the nanoparticle linker and the macrocycle; and
each occurrence of optionally substituted alkyl, optionally substituted alkylenyl, optionally substituted cycloalkyl, optionally substituted cycloalkylenyl, optionally substituted heteroalkyl, optionally substituted heteroalkylenyl, optionally substituted heterocycloalkyl, optionally substituted heterocycloalkylenyl, optionally substituted aryl, optionally substituted arylenyl, optionally substituted heteroaryl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

Embodiment 3 provides the hydrogel composition of Embodiment 2, wherein L^{1a} and L^{1b} are each independently

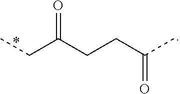

Embodiment 4 provides the hydrogel composition of Embodiment 2 or 3, wherein X^{1a} and X^{1b} are each independently —NH—.

Embodiment 5 provides the hydrogel composition of any one of Embodiments 2-4, wherein L² is

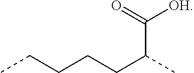

Embodiment 6 provides the hydrogel composition of any one of Embodiments 2-5, wherein the nanoparticle linker is

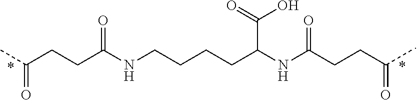

Embodiment 7 provides the hydrogel composition of any one of Embodiments 1-6, wherein each of the plurality of macrocycles is independently selected from the group consisting of a cyclodextrin, a pillar[n]arene, a calix[n]arene, and a cucurbit[n]uril.

Embodiment 8 provides the hydrogel composition of Embodiment 7, wherein each cyclodextrin is independently selected from the group consisting of α-cyclodextrin, β-cyclodextrin, and γ-cyclodextrin.

Embodiment 9 provides the hydrogel composition of Embodiment 7, wherein each cyclodextrin is independently selected from the group consisting of 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, and an aminated cyclodextrin.

Embodiment 10 provides the hydrogel composition of Embodiment 7, wherein each nanoparticle linker is covalently conjugated to a primary hydroxyl of the cyclodextrin.

Embodiment 11 provides the hydrogel composition of any one of Embodiments 1-10, wherein the hydrophilic polymer core is selected from the group consisting of a hyaluronic acid derivative, a dextran derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glycol)fumarate) derivative, a poly(vinyl alcohol) derivative, and a poly(vinyl acid) derivative.

Embodiment 12 provides the hydrogel composition of any one of Embodiments 1-11, wherein each hydrophobic substituent is independently selected from the group consisting of adamantyl, cyclohexyl, benzyl, azobenzyl, and ferrocenyl.

Embodiment 13 provides the hydrogel composition of any one of Embodiments 1-12, wherein the functionalized polymer is a compound of Formula (II):

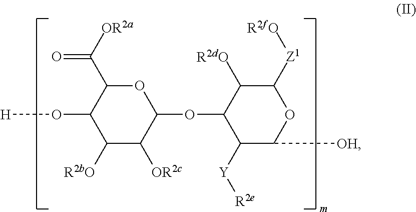

wherein:
each occurrence of R^{2a}, R^{2b}, R^{2c}, R^{2d}, R^{2e}, and R^{2f} is independently selected from the group consisting of R³, H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl,
   wherein the compound of Formula (II) comprises n occurrences of R³,
   wherein 0<n≤m, and
   wherein no more than one of R^{2a}, R^{2b}, R^{2c}, R^{2d}, R^{2e}, and R^{2f} in each repeated unit of the compound Formula (II) is R³;
R³ is

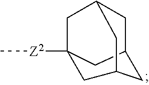

Y is selected from the group consisting of —N(R′)— and —O—;
Z¹ and Z² are each independently selected from the group consisting of -(optionally substituted C₁-C₆ alkylenyl)-, -(optionally substituted C₁-C₆ heteroalkylenyl)-, —C(═O)(optionally substituted C₁-C₆ alkylenyl)-, —C(═O)(optionally substituted C₁-C₆ heteroalkylenyl)-, -(optionally substituted C₁-C₆ alkylenyl)C(═O)—, and -(optionally substituted C₁-C₆ heteroalkylenyl)C(═O)—;
m is an integer ranging from 2 to 1000;
n is an integer ranging from 1 to 999; and
each occurrence of R^{I} and R^{II} is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl.

Embodiment 14 provides the hydrogel composition of Embodiment 13, wherein the compound of Formula (II) is a compound of Formula (IIa):

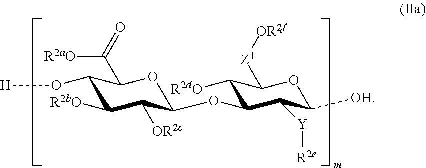

Embodiment 15 provides the hydrogel composition of Embodiment 13 or 14, wherein each of the following apply:
(a) each occurrence of R^{2a}, R^{2b}, R^{2c}, R^{2d}, and R^{2e} is independently selected from the group consisting of H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl; and
(b) each occurrence of R^{2f} is independently selected from the group consisting of H and R³;

Embodiment 16 provides the hydrogel composition of any one of Embodiments 13-15, wherein each occurrence of R^{2a}, R^{2b}, R^{2c}, and R^{2d} is independently H.

Embodiment 17 provides the hydrogel composition of any one of Embodiments 13-16, wherein each occurrence of R^{2e} is independently C(═O)Me.

Embodiment 18 provides the hydrogel composition of any one of Embodiments 13-17, wherein each occurrence of Y is independently —NH—.

Embodiment 19 provides the hydrogel composition of any one of Embodiments 13-18, wherein each occurrence of Z¹ is independently —CH₂—.

Embodiment 20 provides the hydrogel composition of any one of Embodiments 13-19, wherein each occurrence of Z² is —C(═O)CH₂—.

Embodiment 21 provides the hydrogel composition of any one of Embodiments 13-20, wherein each occurrence of R³ is independently

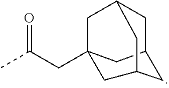

Embodiment 22 provides the hydrogel composition of any one of Embodiments 13-21, wherein m is an integer ranging from 200 to 1000.

Embodiment 23 provides the hydrogel composition of any one of Embodiments 13-22, wherein n and m have a ratio of about 1:10 to about 1:2 (n:m).

Embodiment 24 provides the hydrogel composition of any one of Embodiments 1-23, wherein the therapeutic agent is a small molecule therapeutic agent.

Embodiment 25 provides the hydrogel composition of any one of Embodiments 1-24, wherein the therapeutic agent is selected from the group consisting of an anti-inflammatory agent and a pro-inflammatory agent.

Embodiment 26 provides the hydrogel composition of Embodiment 25, wherein the anti-inflammatory agent is selected from the group consisting of celastrol, piclamilast, ciglitazone, celecoxib, betamethasone, pravastatin, aspirin, ketorolac, lornoxicam, cortisone, pioglitazone, prednisolone, triamcinolone, methylprednisolone, desoximetasone, dexamethasone, Fluvastatin, lovastatin, simvastatin, atorvastatin, rosuvastatin, gemfibrozil, troglitazone, rolipram, fenofibrate, rosiglitazone, apremilast, cilomilast, crisoborole, roflumilast, iloprost, colchicine, quercetin, JSH-23, tuftsin, resolving D1, sivelestat, doxycycline, methotrexate, and vortioxetine.

Embodiment 27 provides the hydrogel composition of Embodiment 25, wherein the pro-inflammatory agent is selected from the group consisting of GW2580, CEP32496, BLZ945, OSI930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, NIK12192, trichostatin A, IBET151, TMP195, BYL719, GDC0941, BKM120, imiquimod, gardiquimod, resiquimod (R848), motolimod, and GS9620.

Embodiment 28 provides the hydrogel composition of any one of Embodiments 1-27, wherein the nanoparticle has a diameter ranging from about 50 nm to about 100 nm, optionally wherein the nanoparticle has a diameter of about 80 nm.

Embodiment 29 provides the hydrogel composition of any one of Embodiments 1-28, wherein the nanoparticle comprises β-cyclodextrin succinate crosslinked with lysine, optionally wherein the β-cyclodextrin succinate and lysine have a ratio of about 1:1.

Embodiment 30 provides the hydrogel composition of any one of Embodiments 1-29, wherein the functionalized polymer has a degree of monomer functionalization ranging from about 5% to about 50%, optionally wherein the functionalized polymer has a degree of monomer functionalization selected from the group consisting of about 10%, about 18%, and about 43%.

Embodiment 31 provides the hydrogel composition of any one of Embodiments 1-30, wherein the functionalized polymer comprises about 1% (w/v) to about 15% (w/v) of the hydrogel composition, optionally wherein the functionalized polymer comprises about 2.5% (w/v), about 5.0% (w/v), 7.5% (w/v), or about 10.0% (w/v) of the hydrogel composition.

Embodiment 32 provides the hydrogel composition of any one of Embodiments 1-31, wherein the nanoparticle and the functionalized polymer have a ratio in the hydrogel composition ranging from about 10:1 to about 1:10 (nanoparticle:functionalized polymer), optionally wherein the nanoparticle and the functionalized polymer have a ratio in the hydrogel composition of about 0.5:1.0, about 1.0:1.0, or about 1.5:1 (nanoparticle:functionalized polymer).

Embodiment 33 provides the hydrogel composition of any one of Embodiments 1-32, wherein the therapeutic agent has a concentration in the hydrogel composition of about 5 μM.

Embodiment 34 provides a method for treating, preventing, and/or ameliorating a disease or disorder in a subject, the method comprising administering to the subject the hydrogel composition of any one of Embodiments 1-33.

Embodiment 35 provides the method of Embodiment 34, wherein the disease or disorder is at least one selected from the group consisting of an inflammatory disease or disorder, a focal tissue injury, an autoimmune disease and/or associated tissue dysfunction, cancer, cytokine storm, and sepsis.

Embodiment 36 provides the method of Embodiment 35, wherein the inflammatory disease or disorder is selected from the group consisting of cardiovascular disease, diabetic wounds, osteoarthritis, inflammatory bowel disease, and colitis.

Embodiment 37 provides the method of Embodiment 36, wherein the cardiovascular disease is at least one selected from the group consisting of atherosclerosis, myocarditis, endocarditis, myocardial infarction, heart failure, stroke, aneurism, aortic dissection, peripheral arterial disease, congenital defects, and valvular disease.

Embodiment 38 provides the method of any one of Embodiments 34-37, wherein the hydrogel composition is locally administered to an inflamed tissue or organ, optionally wherein the inflamed tissue or organ is the heart.

Embodiment 39 provides a method for delivering a therapeutic agent to a macrophage in a subject, the method comprising administering to the subject the hydrogel composition of any one of Embodiments 1-33.

Embodiment 40 provides a method for reducing or inhibiting pro-inflammatory (M1-like) behavior of a macrophage in a subject, the method comprising administering to the subject the hydrogel composition of any one of Embodiments 1-33.

Embodiment 41 provides a method for promoting pro-healing (M2-like) behavior of a macrophage in a subject, the method comprising administering to the subject the hydrogel composition of any one of Embodiments 1-33.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A hydrogel composition comprising:
(a) a nanoparticle comprising a plurality of macrocycles, wherein each of the macrocycles is independently covalently linked, either directly or indirectly, to at least one nanoparticle linker;
(b) a functionalized polymer comprising a hydrophilic polymer core substituted with a plurality of independently selected hydrophobic substituents, wherein at least a portion of the hydrophobic substituents are independently non-covalently associated with one of the macrocycles of the nanoparticle; and
(c) at least one therapeutic agent, wherein the at least one therapeutic agent is non-covalently associated with a macrocycle of the nanoparticle.

2. The hydrogel composition of claim 1, wherein the least one nanoparticle linker independently comprises a moiety of Formula (I): 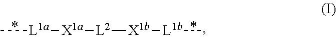
wherein:
L^{1a} and L^{1b} each independently comprise at least one moiety selected from the group consisting of —C(═O)—, -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
L² comprises at least one moiety selected from the group consisting of -(optionally substituted C₁-C₁₂ alkylenyl)-, -(optionally substituted C₃-C₈ cycloalkylenyl)-, -(optionally substituted C₁-C₁₂ heteroalkylenyl)-, -(optionally substituted C₂-C₈ heterocycloalkylenyl)-, -(optionally substituted C₆-C₁₀ arylenyl)-, and -(optionally substituted C₂-C₁₀ heteroarylenyl)-;
X^{1a} and X^{1b} are each independently selected from the group consisting of —N(R′)—, —O—, and —S—;
each occurrence of R¹ is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl;
each bond [symbol] comprises a bond between the nanoparticle linker and the macrocycle; and
each occurrence of optionally substituted alkyl, optionally substituted alkylenyl, optionally substituted cycloalkyl, optionally substituted cycloalkylenyl, optionally substituted heteroalkyl, optionally substituted heteroalkylenyl, optionally substituted heterocycloalkyl, optionally substituted heterocycloalkylenyl, optionally substituted aryl, optionally substituted arylenyl, optionally substituted heteroaryl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

3. The hydrogel composition of claim 2, wherein L^{1a} and L^{1b} are each independently

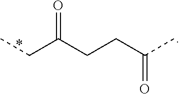

4. The hydrogel composition of claim 2, wherein X^{1a} and X^{1b} are each independently —NH—.

5. The hydrogel composition of claim 2, wherein L² is

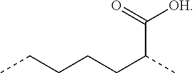

6. The hydrogel composition of claim 2, wherein the nanoparticle linker is

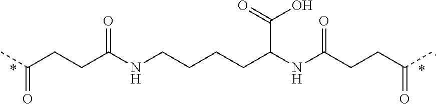

7. The hydrogel composition of claim 1, wherein each of the plurality of macrocycles is independently selected from the group consisting of a cyclodextrin, a pillar[n]arene, a calix[n]arene, and a cucurbit[n]uril.

8. The hydrogel composition of claim 7, wherein each cyclodextrin is independently selected from the group consisting of α-cyclodextrin, β-cyclodextrin, and γ-cyclodextrin.

9. The hydrogel composition of claim 7, wherein each cyclodextrin is independently selected from the group consisting of 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, and an aminated cyclodextrin.

10. The hydrogel composition of claim 7, wherein each nanoparticle linker is covalently conjugated to a primary hydroxyl of the cyclodextrin.

11. The hydrogel composition of claim 1, wherein the hydrophilic polymer core is selected from the group consisting of a hyaluronic acid derivative, a dextran derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glycol)fumarate) derivative, a poly(vinyl alcohol) derivative, and a poly(vinyl acid) derivative.

12. The hydrogel composition of claim 1, wherein each hydrophobic substituent is independently selected from the group consisting of adamantyl, cyclohexyl, benzyl, azobenzyl, and ferrocenyl.

13. The hydrogel composition of claim 1, wherein the functionalized polymer is a compound of Formula (II): 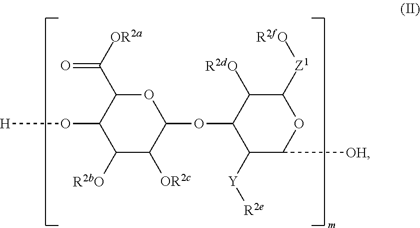
wherein:
each occurrence of R^{2a}, R^{2b}, R^{2c}, R^{2d}, R^{2e}, and R^{2f} is independently selected from the group consisting of R³, H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl,
wherein the compound of Formula (II) comprises n occurrences of R³,
wherein 0<n≤m, and
wherein no more than one of R^{2a}, R^{2b}, R^{2c}, R^{2d}, R^{2e}, and R^{2f} in each repeated unit of the compound Formula (II) is R³;
R³ is 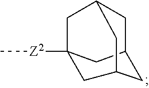
Y is selected from the group consisting of —N(R^{I})— and —O—;
Z¹ and Z² are each independently selected from the group consisting of -(optionally substituted C₁-C₆ alkylenyl)-, -(optionally substituted C₁-C₆ heteroalkylenyl)-, —C(═O)(optionally substituted C₁-C₆ alkylenyl)-, —C(═O)(optionally substituted C₁-C₆ heteroalkylenyl)-, -(optionally substituted C₁-C₆ alkylenyl)C(═O)—, and -(optionally substituted C₁-C₆ heteroalkylenyl)C(═O)—;
m is an integer ranging from 2 to 1000;
n is an integer ranging from 1 to 999; and
each occurrence of R^{I} and R^{II} is independently selected from the group consisting of H, optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl.

14. The hydrogel composition of claim 13, wherein the compound of Formula (II) is a compound of Formula (IIa):

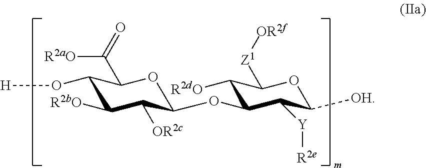

15. The hydrogel composition of claim 13, wherein each of the following apply:
(a) each occurrence of R^{2a}, R^{2b}, R^{2c}, R^{2d}, and R^{2e} is independently selected from the group consisting of H, C(═O)R^{I}, C(═O)OR^{I}, C(═O)N(R^{I})(R^{II}), optionally substituted C₁-C₆ alkyl, optionally substituted C₃-C₈ cycloalkyl, optionally substituted C₁-C₆ heteroalkyl, optionally substituted C₂-C₈ heterocycloalkyl, optionally substituted C₆-C₁₀ aryl, and optionally substituted C₂-C₁₀ heteroaryl; and
(b) each occurrence of R^{2f} is independently selected from the group consisting of H and R³;

16. The hydrogel composition of claim 13, wherein each occurrence of R^{2a}, R^{2b}, R^{2c}, and R^{2d} is independently H.

17. The hydrogel composition of claim 13, wherein each occurrence of R^{2e} is independently C(═O)Me.

18. The hydrogel composition of claim 13, wherein each occurrence of Y is independently —NH—.

19. The hydrogel composition of claim 13, wherein each occurrence of Z¹ is independently —CH₂—.

20. The hydrogel composition of claim 13, wherein each occurrence of Z² is —C(═O)CH₂—.

21. The hydrogel composition of claim 13, wherein each occurrence of R³ is independently

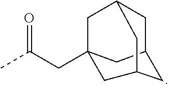

22. The hydrogel composition of claim 13, wherein m is an integer ranging from 200 to 1000.

23. The hydrogel composition of claim 13, wherein n and m have a ratio of about 1:10 to about 1:2 (n:m).

24. The hydrogel composition of claim 1, wherein the therapeutic agent is a small molecule therapeutic agent.

25. The hydrogel composition of claim 1, wherein the therapeutic agent is selected from the group consisting of an anti-inflammatory agent and a pro-inflammatory agent.

26. The hydrogel composition of claim 25, wherein the anti-inflammatory agent is selected from the group consisting of celastrol, piclamilast, ciglitazone, celecoxib, betamethasone, pravastatin, aspirin, ketorolac, lornoxicam, cortisone, pioglitazone, prednisolone, triamcinolone, methylprednisolone, desoximetasone, dexamethasone, Fluvastatin, lovastatin, simvastatin, atorvastatin, rosuvastatin, gemfibrozil, troglitazone, rolipram, fenofibrate, rosiglitazone, apremilast, cilomilast, crisoborole, roflumilast, iloprost, colchicine, quercetin, JSH-23, tuftsin, resolving D1, sivelestat, doxycycline, methotrexate, and vortioxetine.

27. The hydrogel composition of claim 25, wherein the pro-inflammatory agent is selected from the group consisting of GW2580, CEP32496, BLZ945, OSI930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, NIK12192, trichostatin A, IBET151, TMP195, BYL719, GDC0941, BKM120, imiquimod, gardiquimod, resiquimod (R848), motolimod, and GS9620.

28. The hydrogel composition of claim 1, wherein the nanoparticle has a diameter ranging from about 50 nm to about 100 nm, optionally wherein the nanoparticle has a diameter of about 80 nm.

29. The hydrogel composition of claim 1, wherein the nanoparticle comprises β-cyclodextrin succinate crosslinked with lysine, optionally wherein the β-cyclodextrin succinate and lysine have a ratio of about 1:1.

30. The hydrogel composition of claim 1, wherein the functionalized polymer has a degree of monomer functionalization ranging from about 5% to about 50%, optionally wherein the functionalized polymer has a degree of monomer functionalization selected from the group consisting of about 10%, about 18%, and about 43%.

31. The hydrogel composition of claim 1, wherein the functionalized polymer comprises about 1% (w/v) to about 15% (w/v) of the hydrogel composition, optionally wherein the functionalized polymer comprises about 2.5% (w/v), about 5.0% (w/v), 7.5% (w/v), or about 10.0% (w/v) of the hydrogel composition.

32. The hydrogel composition of claim 1, wherein the nanoparticle and the functionalized polymer have a ratio in the hydrogel composition ranging from about 10:1 to about 1:10 (nanoparticle:functionalized polymer), optionally wherein the nanoparticle and the functionalized polymer have a ratio in the hydrogel composition of about 0.5:1.0, about 1.0:1.0, or about 1.5:1 (nanoparticle:functionalized polymer).

33. The hydrogel composition of claim 1, wherein the therapeutic agent has a concentration in the hydrogel composition of about 5 μM.

34. A method for treating, preventing, and/or ameliorating a disease or disorder in a subject, the method comprising administering to the subject the hydrogel composition of claim 1.

35. The method of claim 34, wherein the disease or disorder is at least one selected from the group consisting of an inflammatory disease or disorder, a focal tissue injury, an autoimmune disease and/or associated tissue dysfunction, cancer, cytokine storm, and sepsis.

36. The method of claim 35, wherein the inflammatory disease or disorder is selected from the group consisting of cardiovascular disease, diabetic wounds, osteoarthritis, inflammatory bowel disease, and colitis.

37. The method of claim 36, wherein the cardiovascular disease is at least one selected from the group consisting of atherosclerosis, myocarditis, endocarditis, myocardial infarction, heart failure, stroke, aneurism, aortic dissection, peripheral arterial disease, congenital defects, and valvular disease.

38. The method of claim 34, wherein the hydrogel composition is locally administered to an inflamed tissue or organ, optionally wherein the inflamed tissue or organ is the heart.

39. A method for delivering a therapeutic agent to a macrophage in a subject, the method comprising administering to the subject the hydrogel composition of claim 1.

40. A method for reducing or inhibiting pro-inflammatory (M1-like) behavior of a macrophage in a subject, the method comprising administering to the subject the hydrogel composition of claim 1.

41. A method for promoting pro-healing (M2-like) behavior of a macrophage in a subject, the method comprising administering to the subject the hydrogel composition of claim 1.
